(19) United States
(12) Patent Application Publication
(10) Pub. No.: US 2022/0089531 A1
(43) Pub. Date: Mar. 24, 2022

(54) NEW SALICYLIC ACID DERIVATIVES, PHARMACEUTICALLY ACCEPTABLE SALT THEREOF, COMPOSITION THEREOF AND METHOD OF USE THEREOF

(71) Applicants: THE GOVERNING COUNCIL OF THE UNIVERSITY OF TORONTO, Toronto (CA); Janpix Ltd., London (GB)

(72) Inventors: Patrick Thomas Gunning, Mississauga (CA); Siawash Ahmar, Toronto (CA); David Rosa, Toronto (CA); Gary Tin, Richmond Hill (CA); Mulu Geletu, Mississauga (CA); Ji Sung Park, Milton (CA); Elvin D. De Araujo, Mississauga (CA)

(21) Appl. No.: 17/416,175

(22) PCT Filed: Dec. 20, 2019
(86) PCT No.: PCT/CA2019/051884

§ 371 Date: Jun. 18, 2021

Related U.S. Application Data
(60) Provisional application No. 62/783,741, filed on Dec. 21, 2018.

(51) Int. Cl. C07C 311/19; A61P 35/00

(52) U.S. Cl. CPC C07C 311/19; A61P 35/00

(57) ABSTRACT

The present invention relates to novel compounds, compositions containing same and methods for inhibiting STAT3 and/or STAT5 activity or for the treatment of a cell proliferative disorder such as a cancer using a compound of formula I

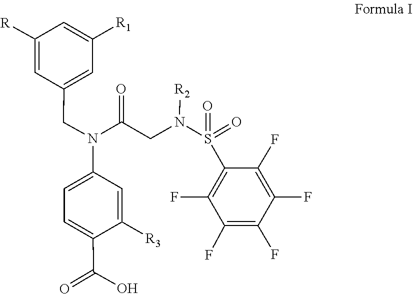

or a pharmaceutically acceptable salt, solvate or hydrate thereof, wherein
R and R₁, different, are selected from the group consisting of

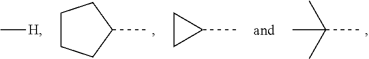

wherein when one of R and R₁ is a —H, the other of R and R₁ is a cyclopentyl moiety,
R₂ is a benzyl substituted with 1-5 halogens, preferably —Cl or —F, and
R₃ is selected from the group consisting of —H or —OH.

### FIELD OF THE DISCLOSURE

The present invention relates to novel salicylic acid derivative compound, compositions containing same and methods inhibiting STAT3 activity or for treating cancer where STAT3/5 are involved, such as in brain, breast, colon, hematologic, lung, ovarian and prostate cancers using said compounds.

### BACKGROUND OF THE DISCLOSURE

STAT3 is persistently activated in over a dozen types of human cancers, including all the major carcinomas, including breast, brain, colon, pancreas, ovarian, and squamous cell carcinomas of head and neck (SCCHN) cancers, and melanomas as well as some hematologic tumors (Bowman T, et al (2000) Oncogene 19, 2474-88, and Darnell, J. E. (2005) Nat. Med. 1 1, 595-596). As such, there is increasing interest in developing anticancer therapies through the inhibition of persistently active STAT3, especially as a strategy to deal with cancers where physicians are looking to improve the outcome and/or where even establishing a satisfactory standard of care has been challenging in terms of patient care, quality of life and outcome.

Glioblastoma (GBM) is considered the most aggressive and lethal of brain cancers, with a median survival after treatment of approximately 15 months. Shockingly, these modest results can only be achieved in the relatively young (i.e., <age 70) and otherwise healthy patients. Older patients with GBM, of which there are many, and those with poor performance status at diagnosis have much shorter survivals following identical therapy. In addition, GBM is occurring with increasing frequency in an aging population. Moreover, unlike the more common cancers, such as those of the lung, breast and colon, GBM is neither preventable, nor detectable at a stage when early treatment might be expected to be substantially more effective. Furthermore, despite decades of intensive research, major improvements in overall survival have remained elusive. As such, the development of therapeutic approaches to meet this unmet need is critical.

Brain tumours have been demonstrated to contain rare subpopulations of brain tumour stem cells (BTSCs), which possess the cardinal stem cell properties of clonogenic self-renewal, multipotency and tumourigenicity. The extensive self-renewal and proliferative capacity of BTSCs coupled with their insensitivity to conventional radio- and chemotherapies suggest that they are integral to the growth and post-treatment recurrence of GBM. As such, BTSCs represent a “reservoir of disease” that require novel therapeutic approaches to effectively eliminate in order to improve the outcome of GBM.

STAT proteins were originally discovered as latent cytoplasmic transcription factors that mediate cytokine and growth factor responses (Darnell, J. E., Jr. (1996) Recent Prog. Norm. Res. 51, 391-403; Darnell, J. E. (2005) Nat. Med. 1 1, 595-596). Seven members of the family, STAT1, STAT2, STAT3, STAT4, STAT5a and STAT5b, and STATE, mediate several physiological effects including growth and differentiation, survival, development and inflammation. STATs are SH2 domain-containing proteins. Upon ligand binding to cytokine or growth factor receptors, STATs become phosphorylated on critical Tyr residue (Tyr705 for STAT3) by growth factor receptors, cytoplasmic Janus kinases (Jaks) or Src family kinases. Two phosphorylated and activated STAT monomers dimerize through reciprocal pTyr-SH2 domain interactions, translocate to the nucleus, and bind to specific DNA-response elements of target genes, thereby inducing gene transcription (Darnell, J. E., Jr. (1996) Recent Prog. Norm. Res. 51, 391-403; Darnell, J. E. (2005) Nat. Med. 1 1, 595-596). In contrast to normal STAT signaling, many human solid and hematological tumors harbor aberrant STAT3 activity (Turkson, J. Expert Opin. Ther. Targets 2004, 8, 409-422; Darnell, J. E., Jr. (1996) Recent Prog. Norm. Res. 51, 391-403; Darnell, J. E. (2005) Nat. Med. 11(6), 595-596; Bowman, T. et al. (2000) Oncogene 19(21), 2474-2488; Buettner, et al. (2002) Clin. Cancer Res. 8(4), 945-954; Yu, H. and Jove. R. (2004) Nat. Rev. Cancer 4(2), 97-105; Haura, E. B., et al. (2005) Nat. Clin. Pract. Oncol. 2(6), 315-324).

Of note, STAT3 protein is one of seven family members of the STAT family of transcription factor proteins. STAT3 is activated through phosphorylation of a tyrosine 705 (Y705) that initiates complexation of two phosphorylated STAT3 monomers (pSTAT3). pSTAT3 homo-dimers are mediated through reciprocal STAT3 Src Homology 2 (SH2) domain-pY705 STAT3 interactions. pSTAT3:pSTAT3 homodimers translocate to the nucleus and bind DNA, promoting STAT3 target gene transcription. Targeting STAT3 has been previously achieved with dominant negative constructs, oligonucleotides or, most commonly, phosphopeptidic agents that mimic the native pY705 containing binding sequence. Unfortunately, these inhibitors are rapidly degraded in vivo, which limits their use in the clinic. To circumvent these problems, small molecule STAT3 inhibitors were designed for treatment of cancers harboring hyperactivated STAT3 protein. Acid-based inhibitors have been identified in WO2012/018868 that potently and selectively block STAT3 dimerization and DNA-binding activity, namely, compound 450, also referred to as BP-1-102 (sometimes referred to as compound 1 herein). Compound 450 in WO2012018868 potently suppresses multiple oncogenic properties in diverse cultured cancer cells (breast, lung, pancreatic, prostate, lung), including: cell proliferation, anchorage-independent cell growth, migration, invasion and motility. It is selective for STAT3, with over 10-fold less binding to 93% homologous STAT protein, STAT1. It showed little or no effect on phosphorylation of Shc, Src, Jak-1/2, Erk1/2 or Akt and had no effect on non-transformed cells (NIH 3T3 cells, STAT3 null mouse embryo fibroblasts, or mouse thymus stromal cells, nor does it affect transformed cells that do not harbor activated STAT3). Moreover, BP-1-102 exhibited striking anti-tumor effects in vivo in murine xenograft models of lung or breast cancer resulting in dramatic regression in tumor volumes. Western blots of residual tumors from treated mice showed repression in pSTAT3, cMyc, Cyclin D1, Bc1-xL, Survivin, and VEGF in a dose-dependent manner. Still, WO2013/177534 teaches alternative derivative compound, inhibiting STAT3 activity or for treating cancer where STAT3/5 are involved.

Moreover, genetic and other molecular evidence reveals persistent Tyr phosphorylation of STAT3 is mediated by aberrant upstream Tyr kinases and shows cancer cell requirement for constitutively-active and dimerized STAT3 for tumor maintenance and progression. Thus, in numerous proof-of-concept studies (Turkson, J., et al. Mol. Cancer Ther. 2004, 3(3), 261-269; Turkson, J., et al. J. Biol. Chem. 2001, 276(48), 45443-45455; Siddiquee, K.; et al. Proc. Natl. Acad. Sci. U.S.A. 2007, 104, 7391-7396; Turkson, J.; et al. Mol. Cancer Ther. 2004, 3, 1533-1542; and Turkson, J.; et al. J. Biol. Chem. 2005, 280(38), 32979-32988), inhibition of STAT3 activation or disruption of dimerization induces cancer cell death and tumor regression. Small-molecule STAT3 inhibitors thus provide tools for probing the molecular dynamics of the cellular processing of STAT3 to understand STAT3's role as a signaling intermediate and a molecular mediator of the events leading to carcinogenesis and malignant progression. Moreover, since the STAT3 pathway is a key oncogenic driver in over a dozen types of human cancers, including all the major carcinomas, including breast, brain, colon, pancreas, ovarian, and squamous cell carcinomas of head and neck (SCCHN) cancers, and melanomas as well as some hematologic tumors (Bowman T, et al (2000) Oncogene 19, 2474-88, and Darnell, J. E. (2005) Nat. Med. 1 1, 595-596) the direct inhibition of STAT3 would provide a molecularly targeted route for effectively managing these cancers and especially aggressive forms such as GBM.

In a seminal paper, Carro et al. (Nature, 463(7279): 318-325, 2010) demonstrated that the Signal transducer and activator of transcription 3 (STAT3) gene abnormally active in GBM, is a critically important mediator of tumour growth and therapeutic resistance in GBM. Poorly treated brain cancers such as gliomas, astrocytomas and glioblastomas harbor constitutively activated STAT3. In addition, a growing body of recent evidence gathered using a variety of different small molecules that indirectly inhibit STAT3 by targeting upstream molecules such as the JAK family members, strongly suggest that STAT3 signaling is crucial for the survival and proliferation of BTSCs and GBM both in vitro and in vivo. However, due to their broad targeting nature existing drugs for treating GBM have limited translational potential due to numerous side effects. Hence, drugs with the ability to more specifically block STAT3 activity may provide effective treatment for GBM patients.

STAT5 signaling, like STAT3 signaling, is transiently activated in normal cells and is deactivated by a number of different cytosolic and nuclear regulators, including phosphatases, SOCS, PIAS, and proteasomal degradation. Like STAT3, STAT5 has gained notoriety for its aberrant role in human cancers and tumorigenesis, having been found to be constitutively activated in many cancers, including those of the breast, liver, prostate, blood, skin, head and neck. (Muller, J., et al. ChemBioChem 2008, 9, 723-727). In cancer cells, STAT5 is routinely constitutively phosphorylated which leads to the aberrant expression of STAT5 target genes resulting in malignant transformation. Cancer cells harbouring persistently activated STAT5 over express anti-apoptotic proteins, such as Bcl-xL, Myc and MCL-1, conferring significant resistance to natural apoptotic cues and administered chemotherapeutic agents. Of particular interest, STAT5 has been identified as a key regulator in the development and progression of acute myelogenic (AML) and acute lymphoblastic leukemias (ALL; Gouilleux-Gruart, V., et al. Leukemia and Lymphoma 1997, 28, 83-88; Gouilleux-Gruart, V., et al. Blood 1996, 87, 1692-1697; Weber-Nordt, R. M., et al. Blood 1996, 88, 809-816). Moreover, inhibitors of upstream STAT5 activators (such as J A and FLT3) have been shown to exhibit promising anti-cancer properties (Pardanani, A., et al. Leukemia 2011, 25, 218-225; Quintas—Cardama, A., et al. Nature Reviews Drug Discovery 2011, 10, 127-140).

It should be noted that, medical benefits through the inhibition of STAT3/5 are not limited to the various forms of cancer described herein where these targets are constituatively activated, but would also be applicable to treating other conditions where these pathways are know to play a key role, such as, but not limited to autoimmune disorders (Harris, T. J.; et al Immunol. (2007) 179(7): 4313-4317), inflammation associated with arthritis (Miyamoto. T, et al, Arthritis Research & Therapy (2012), 14(Suppl 1):P43), inflammatory bowel disease (IBD) (World J Gastroenterol.(2008) 14(33): 5110-5114), diabetes (Mashili, F.; et al (2013) Diabetes 62(2), 457-465), irritable bowel syndrome (IBS); kidney disease (Weimbs, T., (2013) JAK-STAT, 2(2), 0-1) and organ transplant (Debonera, F.; et al (2001) J. Surg. Res. 96(2), 289-295).

Despite advances in drug discovery directed to identifying inhibitors of STAT protein activity, there is still a scarcity of compounds that are both potent, efficacious, and selective activators of STAT3 and STAT5 and also effective in the treatment of cancer and other diseases associated with dysfunction in STAT3, STAT5 or both proteins, and diseases in which one or both of STAT3 and STAT5 is involved. Moreover, there is still a need for optimization of potency and reduced pharmacokinetic labilities of existing compounds. These needs and other needs are satisfied by the present invention.

### SUMMARY

In accordance with the purpose(s) of the invention, as embodied and broadly described herein, the invention, in one aspect, relates to compounds useful as inhibitors of STAT3.

In a further aspect, the disclosed compounds and products of disclosed methods of making, or a pharmaceutically acceptable salt, hydrate, solvate, or polymorph thereof, are modulators of STAT3 and/or STAT5 activity, methods of making same, pharmaceutical compositions comprising same, and methods of treating disorders associated with a STAT3 activity dysfunction using same.

In a still further aspect, the present invention relates to compounds that bind to STAT3 protein and negatively modulate STAT3 activity.

In a further aspect, the present invention relates to compounds that bind to STAT5 protein and negatively modulate STAT5 activity.

Also disclosed are pharmaceutical compositions comprising a therapeutically effective amount of a disclosed compound and a pharmaceutically acceptable carrier.

Disclosed are methods for the treatment of a disorder associated with STAT3/STAT5 activity dysfunction, preferably hyperactivity or over-expression, in a mammal comprising the step of administering to the mammal a therapeutically effective amount of a disclosed compound, or a pharmaceutically acceptable salt, hydrate, solvate, or polymorph thereof.

Also disclosed are methods for inhibition of STAT3 and/or STAT5 activity in a mammal comprising the step of administering to the mammal a therapeutically effective amount of least one disclosed compound, or a pharmaceutically acceptable salt, hydrate, solvate, or polymorph thereof.

Also disclosed are methods for inhibiting STAT3 and/or STAT5 activity in at least one cell, comprising the step of contacting the at least one cell with an effective amount of least one disclosed compound, or a pharmaceutically acceptable salt, hydrate, solvate, or polymorph thereof.

Also disclosed are uses of at least one disclosed compound, or a pharmaceutically acceptable salt, hydrate, or solvate thereof.

In one aspect, there is provided a compound of formula I as defined herein.

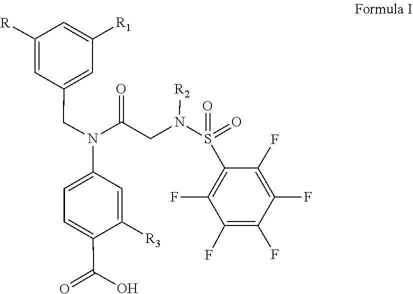

or a pharmaceutically acceptable salt, solvate or hydrate thereof, wherein
R is different from R₁ and both R and R₁ are selected from the group consisting of:

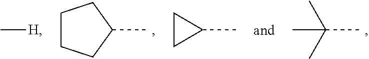

wherein when one of R and R₁ is a —H, the other of R and R₁ is a cyclopentyl moiety,
R₂ is a benzyl substituted with 1-5 halogens, preferably —Cl or —F, and
R₃ is selected from the group consisting of H or OH.

In a further aspect, the invention relates to pharmaceutical compositions comprising a pharmaceutically acceptable carrier and an effective amount of a disclosed compound, or a pharmaceutically acceptable salt, hydrate, or solvate thereof.

In another aspect of the disclosure, there is provided a pharmaceutical composition comprising a compound as defined herein or a pharmaceutically acceptable salt, hydrate or solvate thereof, and an acceptable excipient.

In another aspect of the disclosure, there is provided a method for inhibiting STAT3 and/or STAT5 activity, comprising administering a therapeutically effective amount of a compound as defined herein or a pharmaceutically acceptable salt, solvate or hydrate thereof, to a patient.

In yet another aspect of the disclosure, there is provided a method for treating or preventing cancer associated with STAT3/STAT5 activity dysfunction (preferably hyperactivity thereof or over-expression of same) comprising administering a therapeutically effective amount of a compound as defined herein, or a pharmaceutically acceptable salt, solvate or hydrate thereof, to a patient. In alternative aspect, the cancer is from solid or hematological tumors. Still in other aspect, the cancer is one harbouring activated STAT3 and/or STAT5. Such cancer can be for example breast, liver, prostate, blood, skin, head, neck cancer, glioblastoma or acute myelogenic (AML) and acute lymphoblastic leukemias.

In another aspect of the disclosure, there is provided the use of a compound as defined herein or a pharmaceutically acceptable salt, solvate or hydrate thereof, in the manufacture of a medicament for inhibiting STAT3 and/or STAT5 activity.

In another aspect of the disclosure, there is provided the use of a compound as defined herein or a pharmaceutically acceptable salt, solvate or hydrate thereof, in the manufacture of a medicament for treating or preventing cancer harbouring activated STAT3 and/or STAT5, such as cancer from solid or hematological tumors, breast cancer, liver cancer, prostate cancer, blood cancer, skin cancer, head cancer, neck cancer, glioblastoma or acute myelogenic (AML) and acute lymphoblastic leukemias.

In yet another aspect of the disclosure, there is provided the use of a compound as defined herein or a pharmaceutically acceptable salt, solvate or hydrate thereof, for inhibiting STAT3 and/or STAT5 activity.

In another aspect of the disclosure, there is provided the use of a compound as defined herein or a pharmaceutically acceptable salt, solvate or hydrate thereof, for treating or preventing cancer harbouring activated STAT3 and/or STAT5, such as the cancer is from solid or hematological tumors, breast cancer, liver cancer, prostate cancer, blood cancer, skin cancer, head cancer, neck cancer, glioblastoma or acute myelogenic (AML) and acute lymphoblastic leukemiasassociated with STAT3/STAT5 activity dysfunction, such as breast, prostate or brain cancer.

In another aspect of the disclosure, there is provided a pharmaceutical composition as defined herein for use in inhibiting STAT3 and/or STAT5 activity.

In yet another aspect of the disclosure there is provided a pharmaceutical composition as defined herein for use in treating or preventing cancer harbouring activated STAT3 and/or STAT5, such as the cancer is from solid or hematological tumors, breast cancer, liver cancer, prostate cancer, blood cancer, skin cancer, head cancer, neck cancer, glioblastoma or acute myelogenic (AML) and acute lymphoblastic leukemias.

Also disclosed are methods for manufacturing a medicament, comprising combining at least one disclosed compound or at least one disclosed product with a pharmaceutically acceptable carrier or diluent. In a further aspect, the invention relates to the use of a disclosed compound in the manufacture of a medicament for the treatment of a disorder associated with STAT3/STAT5 activity dysfunction (such as hyperactivity or over-expression). In a still further aspect, the invention relates to the use of the disclosed compound in the manufacture of a medicament for the treatment of a cancer harbouring activated STAT3 and/or STAT5, such as the cancer is from solid or hematological tumors, breast cancer, liver cancer, prostate cancer, blood cancer, skin cancer, head cancer, neck cancer, glioblastoma or acute myelogenic (AML) and acute lymphoblastic leukemias.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

The present invention can be understood more readily by reference to the following detailed description of the invention and the Examples included therein.

### BRIEF DESCRIPTION OF THE DRAWINGS

FIG. 1 is a graph illustrating a comparative intrinsic clearance rates between AC-3-19 (prior art compound) and compound I;

FIG. 2A illustrates the chemical structure of JPX-0372 (prior art);

FIG. 2B illustrates intrinsic comparative clearance rates between JPX-0372 and compound I;

FIG. 3A illustrates the chemical structure of JPX-0369 (prior art);

FIG. 3B illustrates intrinsic comparative clearance rates between JPX-0369 and compound I

FIG. 4A illustrates the chemical structure of JPX-0371 (prior art);

FIG. 4B illustrates intrinsic comparative clearance rates between JPX-0371 and compound I;

FIG. 5A illustrates the chemical structure of JPX-0318 (prior art);

FIG. 5B illustrates intrinsic comparative clearance rates between JPX-0318 and compound II; and

FIG. 6 illustrates comparative clearance rates between JPX-0371 and compound I in CD-1 mice dosed at 20 mgs/kg (IP).

### DESCRIPTION OF THE EMBODIMENTS

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, EIZ specification, and the like. One of skill in the art can readily ascertain the structure of a compound if given a name, either by systemic reduction of the compound structure using naming conventions, or by commercially available software, such as CHEMDRAW™ (Cambridgesoft Corporation, U.S.A.).

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

Abbreviations used in the description of the preparation of the compounds of the present disclosure:

### Bu Butyl

CDCl₃ Deuterated chloroform

### DCM Dichloromethane

### DMAP N,N-dimethylaminopyridine

DME 1,2-dimethoxyethane

### DMEM Dulbecco's Modified Eagle Medium

### DMF N,N-Dimethylformamide

DMSO Dimethyl sulfoxide

### Et Ethyl

EtOAc Ethyl acetate
HMQC Heteronuclear multiple quantum coherence
mCPBA meta-chloroperbenzoic acid
HRMS High resolution mass spectrum

### Me Methyl

### MeOH Methanol

### NEt₃ Triethylamine

### NF SI N-fluorobenzenesulfonimide

NMR Nuclear magnetic resonance

### Ph Phenyl

RT Room temperature

### THF Tetrahydofuran

TBAF tetrabutylammonium fluoride
TFA trifluoroacetic acid
TMSBr trimethylsilyl bromide
RBF Round bottom flask

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” means that the subsequently described event or circumstance can or cannot occur, and that the description includes instances where said event or circumstance occurs and instances where it does not.

As used herein, the terms “STAT3,” “signal transducer and activator of transcription 3 (acute-phase response),” and “signal transducer and activator of transcription 3” can be used interchangeably and refer to a a transcription factor encoded by a gene designated in human as the STAT3 gene, which has a human gene map locus of 17q21 and described by Entrez Gene cytogenetic band: 17q21.31; Ensembl cytogenetic band: 17q21.2; and, HGNC cytogenetic band: 17q21. The term STAT3 refers to a human protein that has 770 amino acids and has a molecular weight of about 88,068 Da. The term is inclusive of splice isoforms or variants, and also inclusive of that protein referred to by such alternative designations as: APRF, MGC 16063, Acute-phase response factor, DNA-binding protein APRF, HIES as used by those skilled in the art to that protein encoded by human gene STAT3. The term is also inclusive of the non-human ortholog or homolog thereof.

As used herein, “STAT5,” refers to STAT5A and/or STAT5B. If specific reference to either STAT5A or STAT5B is required, the specific term will be used herein.

As used herein, “STAT5A” and “signal transducer and activator of transcription 5A” can be used interchangeably and refer to a a transcription factor encoded by a gene designated in human as the STAT5A gene, which has a human gene map locus described by Entrez Gene cytogenetic band: 17q1 1.2; Ensembl cytogenetic band: 17q21.2; and, HGNC cytogenetic band: 17q 1 1.2. The term STAT5A refers to a human protein that has 794 amino acids and has a molecular weight of about 90,647 Da. The term is inclusive of splice isoforms or variants, and also inclusive of that protein referred to by such alternative designations as MGF and STAT5 as used by those skilled in the art to that protein encoded by human gene STAT5A. The term is also inclusive of the non-human ortholog or homolog thereof.

As used herein, “STAT5B” and “signal transducer and activator of transcription 5B” can be used interchangeably and refer to a a transcription factor encoded by a gene designated in human as the STAT5B gene, which has a human gene map locus described by Entrez Gene cytogenetic band: 17q1 1.2; Ensembl cytogenetic band: 17q21.2; and, HGNC cytogenetic band: 17q1 1.2. The term STAT5A refers to a human protein that has 787 amino acids and has a molecular weight of about 89,866 Da. The term is inclusive of splice isoforms or variants, and also inclusive of that protein referred to by such alternative designations as transcription factor STAT5B as used by those skilled in the art to that protein encoded by human gene STAT5A. The term is also inclusive of the non-human ortholog or homolog thereof.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. In one aspect, the subject is a mammal. A patient refers herein to a subject afflicted with cancer, preferably glioblastoma. The term “patient” includes human and veterinary subjects.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein. For example, “diagnosed with a disorder treatable by STAT3 inhibition” means having been subjected to a physical examination by a person of skill, for example, a physician, and found to have a condition that can be diagnosed or treated by a compound or composition that can inhibit or negatively modulate STAT3. As a further example, “diagnosed with a need for inhibition of STAT3” refers to having been subjected to a physical examination by a person of skill, for example, a physician, and found to have a condition characterized by a dysfunction in STAT3 activity. Such a diagnosis can be in reference to a disorder, such as an oncological disorder or disease, cancer and/or disorder of uncontrolled cellular proliferation and the like, as discussed herein. For example, the term “diagnosed with a need for inhibition of STAT3 activity” refers to having been subjected to a physical examination by a person of skill, for example, a physician, and found to have a condition that can be diagnosed or treated by inhibition of STAT3 activity. For example, “diagnosed with a need for modulation of STAT3 activity” means having been subjected to a physical examination by a person of skill, for example, a physician, and found to have a condition that can be diagnosed or treated by modulation of STAT3 activity, e.g. negative modulation. For example, “diagnosed with a need for treatment of one or more disorder of uncontrolled cellular proliferation associated with STAT3 dysfunction” means having been subjected to a physical examination by a person of skill, for example, a physician, and found to have one or disorders of uncontrolled cellular proliferation, e.g. a cancer, associated with STAT3 dysfunction.

As used herein, the expression “STAT3- or STAT5-dependent cancer” refers to a cancer harboring constitutively activated STAT3 or STAT5.

As used herein, the phrase “identified to be in need of treatment for a disorder,” or the like, refers to selection of a subject based upon need for treatment of the disorder. For example, a subject can be identified as having a need for treatment of a disorder (e.g., a disorder related to STAT3 activity) based upon an earlier diagnosis by a person of skill and thereafter subjected to treatment for the disorder. It is contemplated that the identification can, in one aspect, be performed by a person different from the person making the diagnosis. It is also contemplated, in a further aspect, that the administration can be performed by one who subsequently performed the administration.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

The term “contacting” as used herein refers to bringing a disclosed compound and a cell, target STAT3 protein, or other biological entity together in such a manner that the compound can affect the activity of the target (e.g., spliceosome, cell, etc.), either directly; i.e., by interacting with the target itself, or indirectly; i.e., by interacting with another molecule, co-factor, factor, or protein on which the activity of the target is dependent.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “EC₅₀,” is intended to refer to the concentration of a substance (e.g., a compound or a drug) that is required for 50% agonism or activation of a biological process, or component of a process, including a protein, subunit, organelle, ribonucleoprotein, etc. In one aspect, an EC₅₀ can refer to the concentration of a substance that is required for 50% agonism or activation in vivo, as further defined elsewhere herein. In a further aspect, EC₅₀ refers to the concentration of agonist or activator that provokes a response halfway between the baseline and maximum response.

As used herein, “IC₅₀,” is intended to refer to the concentration of a substance (e.g., a compound or a drug) that is required for 50% inhibition of a biological process, or component of a process, including a protein, subunit, organelle, ribonucleoprotein, etc. In some contexts, an IC₅₀ can refer to the plasma concentration of a substance that is required for 50% inhibition in vivo, as further defined elsewhere herein. More commonly, IC₅₀ refers to the half maximal (50%) inhibitory concentration (IC) of a substance required to inhibit a process or activity in vitro.

As used herein, “STAT3 IC₅₀” refers to the concentration of a substance (e.g., a compound or a drug) that is required for 50% inhibition of a STAT3 activity. In some contexts, an IC₅₀ can refer to the plasma concentration of a substance that is required for 50% inhibition of an in vivo activity or process as further defined elsewhere herein, e.g. tumor growth in an animal or human. In other contexts, STAT3 IC₅₀ refers the half maximal (50%) inhibitory concentration (IC) of a substance or compound required to inhibit a process or activity an in vitro context, e.g. a cell-free or cell-based assay. For example, the STAT3 IC₅₀ can be in the context of the half-maximal concentration required to inhibit cell growth. As discussed below, the response is measured in a cell-line with aberrant STAT3 activity. Alternatively, the response is measured in a cell-line with persistently active STAT3. The response can be determined using a cell-line derived from a human breast cancer, human pancreatic cancer, and human prostate cancer. For example, the response can be measured in a cell-line selected from MDA-MB-231, Panc-1, and DU-145. Cell-lines transfected with specific genes can also be used. For example, the response can be measured in a cell-line transfected with v-Src. Alternatively, the cell-line transfected with v-Src is a permanent cell-line. In some cases, the STAT3 IC₅₀ is the half-maximal concentration required to inhibit STAT3 activity in a cell-free assay, e.g. an electrophoretic mobility shift assay (“EMSA”). Alternatively, the STAT3 IC₅₀ is the half-maximal concentration required to inhibit cell-growth, cell viability or cell migration activity.

As used herein, the term “STAT3 K_{D}” refers to the binding affinity of a compound or substance for the STAT3 determined in an in vitro assay. The K_{D} of a substance for a protein can be determined by a variety of methods known to one skilled in the art, e.g. equilibrium dialysis, analytical ultracentrifugation and surface plasmon resonance (“SPR”) analysis. As typically used herein, STAT3 K_{D} is defined as the ratio of association and dissociation rate constants determined using SPR analysis using purified STAT3 protein.

As used herein, the term “STAT3 Kᵢ” refers to the inhibition constant for the displacement of a STAT3 SH2 probe from STAT3 protein. For example, the STAT3 SH2 can be fluorescence-labelled GpYLPQTV. As described herein, the fluorescence label is 5-carboxyfluorescein, although other suitable fluorescence probes can be used as determined to be useful and convenient by one skilled in the art.

The term “pharmaceutically acceptable” describes a material that is not biologically or otherwise undesirable, i.e., without causing an unacceptable level of undesirable biological effects or interacting in a deleterious manner.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As used herein, the term “pharmaceutically acceptable carrier” refers to sterile aqueous or non-aqueous solutions, dispersions, suspensions or emulsions, as well as sterile powders for reconstitution into sterile injectable solutions or dispersions just prior to use. Examples of suitable aqueous and non-aqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “R”, “R₁”, “R₂”, and “R₃” are used herein as generic symbols to represent various specific substituents. These symbols can be any substituent, not limited to those disclosed herein, and when they are defined to be certain substituents in one instance, they can, in another instance, be defined as some other substituents.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, butyl, n-pentyl, isopentyl, i-pentyl, neopentyl, hexyl, heptyl, octyl, nonyl, decyl, dode cyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like. [0086] This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “polyalkylene group” as used herein is a group having two or more CH₂ groups linked to one another. The polyalkylene group can be represented by the formula—(CH2)ₐ—, where “a” is an integer of from 2 to 500.

The terms “alkoxy” and “alkoxyl” as used herein to refer to an alkyl or cycloalkyl group bonded through an ether linkage; that is, an “alkoxy” group can be defined as—OA¹ where A¹ is alkyl or cycloalkyl as defined above. “Alkoxy” also includes polymers of alkoxy groups as just described; that is, an alkoxy can be a polyether such as—OA¹-OA² or—OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aryl” as used herein is a group that contains any carbon-based aromatic group including, but not limited to, benzene, naphthalene, phenyl, biphenyl, phenoxybenzene, and the like. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. Likewise, the term “non-heteroaryl,” which is also included in the term “aryl,” defines a group that contains an aromatic group that does not contain a heteroatom. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein. The term “biaryl” is a specific type of aryl group and is included in the definition of “aryl.” Biaryl refers to two aryl groups that are bound together via a fused ring structure, as in naphthalene, or are attached via one or more carbon-carbon bonds, as in biphenyl.

The term “aldehyde” as used herein is represented by the formula—C(O)H. Throughout this specification “C(O)” is a short hand notation for a carbonyl group, i.e., C═O.

The terms “amine” or “amino” as used herein are represented by the formula—NA¹A², where A¹ and A² can be, independently, hydrogen or alkyl, cycloalkyl, alkenyl, cycloalkenyl, alkynyl, cycloalkynyl, aryl, or heteroaryl group as described herein.

The term “alkylamino” as used herein is represented by the formula—NH(-alkyl) where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, and the like.

The term “dialkylamino” as used herein is represented by the formula—N(-alkyl)2 where alkyl is a described herein. Representative examples include, but are not limited to, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula—C(O)OH.

The term “ester” as used herein is represented by the formula—OC(O)A¹ or —C(O)OA¹, where A¹ can be alkyl, cycloalkyl, alkenyl, cycloalkenyl, alkynyl, cycloalkynyl, aryl, or heteroaryl group as described herein. The term “polyester” as used herein is represented by the formula—(A¹O—(O)C-A²-C(O)O)ₐ— or—(A¹O(O)C-A²-OC(O))ₐ—, where A¹ and A² can be, independently, an alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein. The term “polyether” as used herein is represented by the formula—(A¹0-A²O)ₐ—, where A¹ and A² can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The term “halide” as used herein refers to the halogens fluorine, chlorine, bromine, and iodine.

The term “heterocycle,” as used herein refers to single and multi-cyclic aromatic or non-aromatic ring systems in which at least one of the ring members is other than carbon. Heterocycle includes azetidine, dioxane, furan, imidazole, isothiazole, isoxazole, morpholine, oxazole, oxazole, including, 1,2,3-oxadiazole, 1,2,5-oxadiazole and 1,3,4-oxadiazole, piperazine, piperidine, pyrazine, pyrazole, pyridazine, pyridine, pyrimidine, pyrrole, pyrrolidine, tetrahydrofuran, tetrahydropyran, tetrazine, including 1,2,4,5-tetrazine, tetrazole, including 1,2,3,4-tetrazole and 1,2,4,5-tetrazole, thiadiazole, including, 1,2,3-thiadiazole, 1,2,5-thiadiazole, and 1,3,4-thiadiazole, thiazole, thiophene, triazine, including 1,3,5-triazine and 1,2,4-triazine, triazole, including, 1,2,3-triazole, 1,3,4-triazole, and the like.

The term “hydroxyl” as used herein is represented by the formula—OH.

The term “ketone” as used herein is represented by the formula A¹C(O)A², where A¹ and A² can be, independently, an alkyl, cycloaikyl, alkenyl, cycloalkenyl, alkynyl, cycloalkynyl, aryl, or heteroaryl group as described herein.

The term “azide” as used herein is represented by the formula—N₃.

The term “nitro” as used herein is represented by the formula—NO₂.

The term “nitrile” as used herein is represented by the formula—CN.

The term “sulfo-oxo” as used herein is represented by the formulas—S(O)A¹S(O)₂A¹, —OS(O)₂A¹, or —OS(O)₂OA¹, where A¹ can be hydrogen or an alkyl, cycloaikyl, alkenyl, cycloalkenyl, alkynyl, cycloalkynyl, aryl, or heteroaryl group as described herein. Throughout this specification “S(O)” is a short hand notation for S=0. The term “sulfonyl” is used herein to refer to the sulfo-oxo group represented by the —S(O)₂A¹, where A¹ can be hydrogen or an alkyl, cycloaikyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an alkyl, cycloaikyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, an alkyl, cycloaikyl, alkenyl, cycloalkenyl, alkynyl, cycloalkynyl, aryl, or heteroaryl group as described herein.

The term “thiol” as used herein is represented by the formula—SH.

“R₁”, “R₂”, “R₃”, “Rₙ” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)₀₋₄R⁰; —(CH₂)₀₋₄OR⁰; —O(CH₂)₀₋₄R⁰, —0—(CH₂)₀₋₄C(O)OR⁰; —(CH₂)₀₋₄CH(OR⁰)₂; —(CH₂)₀₋₄Ph, which may be substituted with R⁰; —(CH₂)₀₋₄O(CH₂)₀₋₁Ph which may be substituted with R⁰; —CH═CHPh, which may be substituted with R⁰; —(CH₂)₀₋₄O(CH₂)₀₋₁-pyridyl which may be substituted with R⁰; —N0₂; —CN; —N₃; —(CH₂)₀₋₄N(R⁰)₂; —(CH₂)₀₋₄N(R⁰)C(O)R⁰; —N(R⁰)C(S)R⁰; —(CH₂)₀₋₄N(R⁰)C(O)NR⁰₂)—N(R⁰)C(S)NR⁰₂; —(CH₂)₀₋₄N(R⁰)C(O)OR⁰—N(R⁰)N(R⁰)C(O)R⁰; —N(R)N(R⁰)C(O)NR⁰₂; —N(R⁰)N(R⁰)C(O)OR⁰; —(CH₂)₀₋₄C(O)R⁰; —C(S)R⁰; —(CH₂)₀₋₄C(O)OR⁰, —(CH₂)₀₋₄C(O)SR⁰; —(CH₂)₀₋₄C(O)OsiR⁰₃; —(CH₂)₀₋₄OC(O)R⁰; —OC(O)(CH₂)₀₋₄SR—; SC(S)SR⁰; —(CH₂)₀₋₄SC(O)R⁰; —(CH₂)₀₋₄C(O)NR⁰₂; —C(S)NR⁰₂; —C(S)SR⁰; —SC(S)SR⁰, —(CH₂)₀₋₄OC(O)NR⁰₂)—C(O)N(OR⁰R⁰; —C(O)C(O)R⁰; —C(O)CH₂C(O)R⁰; —C(NOR⁰)R⁰; —(CH₂)₀₋₄S SR⁰; —(CH₂)₀₋₄S(O)₂R⁰; —(CH₂)₀₋₄S(O)₂OR⁰; —(CH₂)₀₋₄OS(O)₂R⁰; —S(O)₂NR⁰₂; —(CH₂)₀₋₄S(0)R⁰; —N(R⁰)S(O)₂NR⁰₂; —N(R⁰)S(O)₂R⁰; —N(OR⁰)R⁰; —C(NH)NR⁰₂; —P(O)₂R⁰; —P(O)R⁰₂; —OP(O)R⁰₂; —OP(O)(OR⁰)₂; SiR⁰₃; —(C₁₋₄ straight or branched)alkylene)O—N(R⁰)₂; or —(C₁₋₄ straight or branched)alkylene)C(O)ON(R⁰)₂, wherein each R⁰ may be substituted as defined below and is independently hydrogen, C₁₋₆ aliphatic, —CH₂Ph, —O(CH₂)₀₋₁Ph, —CH₂—(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R⁰, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R⁰ (or the ring formed by taking two independent occurrences of R⁰ together with their intervening atoms), are independently halogen, —(CH₂)₀₋₂R*, -(haloR*), —(CH₂)₀₋₂OH, —(CH₂)₀₋₂OR*, —(CH₂)₀₋₂CH(OR*)₂; —O(haloR*), —CN, —N₃, —(CH₂)₀₋₂C(O)R*, —(CH₂)₀₋₂C(O)OH, —(CH₂)₀₋₂C(O)OR*, —(CH₂)₀₋₂SR*, —(CH₂)₀₋₂SH, —(CH₂)₀₋₂NH₂, —(CH₂)₀₋₂NHR*, —(CH₂)₀₋₂NR*₂, —NO₂, —SiR*₃, —OSiR*₃, —C(O)SR*, —(C₁₋₄ straight or branched alkylene)C(O)OR*, or —SSR* wherein each R* is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R* include =0 and ═S.

Suitable divalent substituents on a saturated carbon atom of an “optionally substituted” group include the following: =0, ═S, ═NNR*₂, ═NNHC(O)R*═NNHC(O)OR*, ═NNHS(O)₂R*, ═NR*, ═NOR*—O(C(R*₂))₂₋₃O— or —S(C(R*₂))₂₋₃S— wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R*, -(haloR*), —OH, —OR*, —O(haloR*), —CN, —C(O)OH, —C(O)OR*, —NH₂, —NHR*, —NR*₂, or —NO₂, wherein each R* is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R⁺, —NR⁺₂, —C(O)R⁺, —C(O)OR⁺, —C(O)C(O)R⁺, —C(O)CH₂C(O)R⁺, —S(O)₂R⁺, —S(O)₂NR⁺₂, —C(S)NR⁺₂, —C(NH)NR⁺₂, or —N(R⁺)S(O)₂R⁺; wherein each R⁺ is independently hydrogen, C₁₋₆ aliphatic which may be substituted as defined below, unsubstituted—OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R⁺, taken together with their intervening atom(s) form an unsubstituted 3-12-membered saturated, partially unsaturated, or aryl mono- or bicyclic ring having 0-4 heteroatoms independently selected from nitrogen, oxygen or sulfur.

Suitable substituents on the aliphatic group of R* are independently halogen, —R*, -(haloR*), —OH, —OR*, —O(haloR*), —CN, —C(O)OH, —C(O)OR*, —NH₂, —NHR*, —NR*₂, or —NO₂, wherein each R* is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “leaving group” refers to an atom (or a group of atoms) with electron withdrawing ability that can be displaced as a stable species, taking with it the bonding electrons. Examples of suitable leaving groups include halides—including chloro, bromo, and iodo—and pseudohalides (sulfonate esters)—including triflate, mesylate, tosylate, and brosylate. It is also contemplated that a hydroxyl moiety can be converted into a leaving group via Mitsunobu reaction.

The terms “hydrolysable group” and “hydrolysable moiety” refer to a functional group capable of undergoing hydrolysis, e.g., under basic or acidic conditions. Examples of hydrolysable residues include, without limitation, acid halides, activated carboxylic acids, and various protecting groups known in the art (see, for example, “Protective Groups in Organic Synthesis,” T. W. Greene, P. G. M. Wuts, Wiley-Interscience, 1999).

The term “organic residue” defines a carbon containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

A very close synonym of the term “residue” is the term “radical,” which as used in the specification and concluding claims, refers to a fragment, group, or substructure of a molecule described herein, regardless of how the molecule is prepared. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkyl sulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

“Inorganic radicals,” as the term is defined and used herein, contain no carbon atoms and therefore comprise only atoms other than carbon. Inorganic radicals comprise bonded combinations of atoms selected from hydrogen, nitrogen, oxygen, silicon, phosphorus, sulfur, selenium, and halogens such as fluorine, chlorine, bromine, and iodine, which can be present individually or bonded together in their chemically stable combinations. Inorganic radicals have 10 or fewer, or preferably one to six or one to four inorganic atoms as listed above bonded together. Examples of inorganic radicals include, but not limited to, amino, hydroxy, halogens, nitro, thiol, sulfate, phosphate, and like commonly known inorganic radicals. The inorganic radicals do not have bonded therein the metallic elements of the periodic table (such as the alkali metals, alkaline earth metals, transition metals, lanthanide metals, or actinide metals), although such metal ions can sometimes serve as a pharmaceutically acceptable cation for anionic inorganic radicals such as a sulfate, phosphate, or like anionic inorganic radical. Inorganic radicals do not comprise metalloids elements such as boron, aluminum, gallium, germanium, arsenic, tin, lead, or tellurium, or the noble gas elements, unless otherwise specifically indicated elsewhere herein.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Inglod-Prelog system can be used to assign the (R) or (S) configuration to a chiral carbon.

Compounds described herein comprise atoms in both their natural isotopic abundance and in non-natural abundance. The disclosed compounds can be isotopically—labelled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁶O, ¹⁷O, ³⁵S, ¹⁸F and ³⁶Cl, respectively. Compounds further comprise prodrugs thereof and pharmaceutically acceptable salts of said compounds or of said prodrugs which contain the aforementioned isotopes and/or other isotopes of other atoms are within the scope of this invention. Certain isotopically-labelled compounds of the present invention, for example those into which radioactive isotopes such as H and C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of the present invention and prodrugs thereof can generally be prepared by carrying out the procedures below, by substituting a readily available isotopically labelled reagent for a non-isotopically labelled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvate or water molecules can combine with the compounds according to the invention to form solvates and hydrates. Unless stated to the contrary, the invention includes all such possible solvates.

The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et. al., The Royal Society of Chemistry, 1889-1896, 2004. Examples of co-crystals include p-toluenesulfonic acid and benzenesulfonic acid.

It is also appreciated that certain compounds described herein can be present as an equilibrium of tautomers. For example, ketones with an α-hydrogen can exist in an equilibrium of the keto form and the enol form.

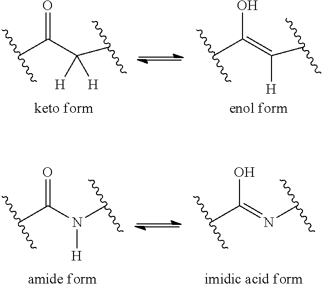 [SMILES: *CC(*)=O ; */C=C(/*)O ; *NC(*)=O ; */N=C(/*)O]

Likewise, amides with an N-hydrogen can exist in equilibrium of the amide form and the imidic acid form. Unless stated to the contrary, the invention includes all such possible tautomers.

It is known that chemical substances form solids which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In some aspects, a structure of a compound can be represented by a formula:

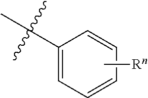

which is understood to be equivalent to a formula:

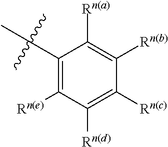

wherein n is typically an integer. That is, Rⁿ is understood to represent five independent substituents, R^{n(a)}, R^{n(b)}, R^{n(c)}, R^{n(d)}, and R^{n(e)}. By “independent substituents,” it is meant that each R substituent can be independently defined. For example, if in one instance R^{n(a)} is halogen, then R^{n(b)} is not necessarily halogen in that instance.

The compounds as defined herein may include a chiral center which gives rise to enantiomers. The compounds may thus exist in the form of two different optical isomers, that is (+) or (−) enantiomers. All such enantiomers and mixtures thereof, including racemic or other ratio mixtures of individual enantiomers, are included within the scope of the invention. The single enantiomer can be obtained by methods well known to those of ordinary skill in the art, such as chiral HPLC, enzymatic resolution and chiral auxiliary derivatization.

It will also be appreciated that the compounds in accordance with the present disclosure can contain more than one chiral centre. The compounds of the present invention may thus exist in the form of different diastereomers. All such diastereomers and mixtures thereof are included within the scope of the invention. The single diastereomer can be obtained by methods well known in the art, such as HPLC, crystalisation and chromatography.

The term “Solvate” means that a compound as defined herein incorporates one or more pharmaceutically acceptable solvents including water to give rise to hydrates. The solvate may contain one or more molecules of solvent per molecule of compound or may contain one or more molecules of compound per molecule of solvent. Illustrative non-limiting examples of hydrates include monohydrate, dihydrate, trihydrate and tetrahydrate or semi-hydrate. In one embodiment, the solvent may be held in the crystal in various ways and thus, the solvent molecule may occupy lattice positions in the crystal, or they may form bonds with salts of the compounds as described herein. The solvate(s) must be “acceptable” in the sense of not being deleterious to the recipient thereof. The solvation may be assessed by methods known in the art such as Loss on Drying techniques (LOD).

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

In one aspect, the invention relates to compounds useful as inhibitors of STAT3/STAT5. In a further aspect, the disclosed compounds and products of disclosed methods of making are modulators of STAT3/STAT5 activity. In various aspects, the present invention relates to compounds that bind to a STAT3 protein and negatively modulate STAT3 activity. In other various aspects, the present invention relates to compounds that bind to a STAT5 protein and negatively modulate STAT5 activity. In a further aspect, the disclosed compounds exhibit inhibition of STAT3/5 activity.

In one aspect, the compounds of the invention are useful in the treatment of cancer associated with STAT3/STAT5 activity dysfunction, such as breast, prostate or brain cancer and glioblastoma, and other diseases in which a STAT3/5 protein is involved, as further described herein.

It is contemplated that each disclosed derivative can be optionally further substituted. It is also contemplated that any one or more derivative can be optionally omitted from the invention. It is understood that a disclosed compound can be provided by the disclosed methods. It is also understood that the disclosed compounds can be employed in the disclosed methods of using.

In one aspect, there is described herein a novel series of compounds that exhibit potent anti-cancer activity, minimal toxicity in normal cells, exemplary metabolic stability in mouse and human hepatocytes, plasma stability in mice. Lead compounds from this series exhibit strong cancer killing potency in acute myeloid leukemia cells, MV4;11 cells with nM IC₅₀s. Two notable examples, compound I (JPX-0431) and compound II (JPX-0432), exhibit ˜6-8-fold greater potency in acute myeloid leukemia cells, MV4;11, than the comparable compound from literature, AC-3-19. The exemplary potency and metabolic stability are attributed to a privileged scaffold including compounds of Formula I, which affords protection of the pentafluorobenzenesulfonamide from attack by biological nucleophiles such as glutathione.

In some aspect, there are disclosed compounds of Formula I or a pharmaceutically acceptable salt and/or solvate thereof:

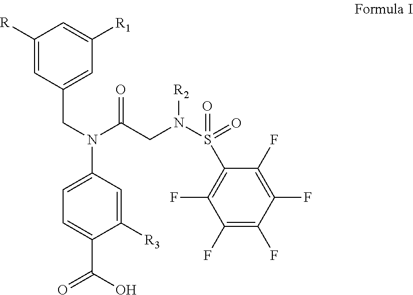

wherein for Formula I:

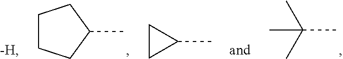

Wherein when one of R and R₁ is a —H, the other of R and R₁ is a cyclopentyl moiety,

R₂ is a benzyl substituted with 1-5 halogens, preferably —Cl or —F, and

R₂ is selected from:

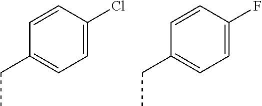

R₃ is selected from the group consisting of —H or —OH.

### Specific Examples

In some aspects, the compound of Formula I is selected from:

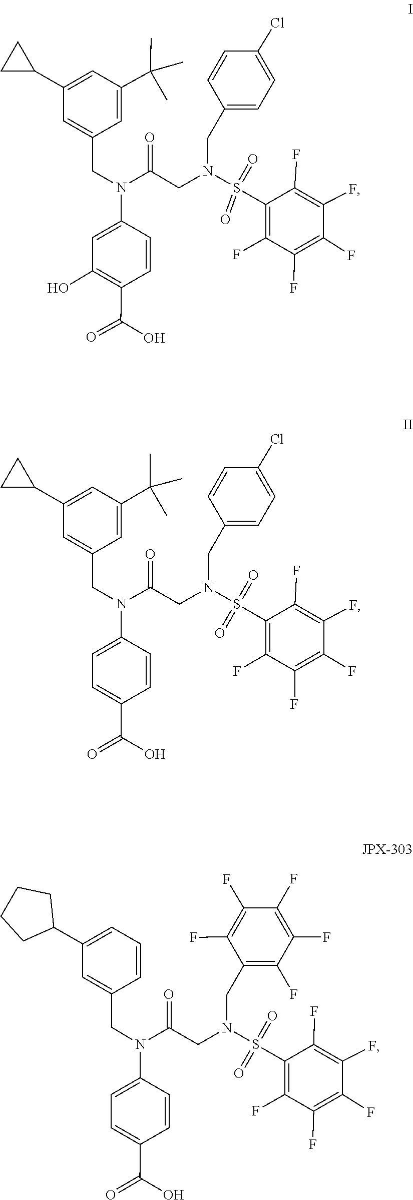 [SMILES: CC(C)(C)c1cc(CN(C(=O)CN(Cc2ccc(Cl)cc2)S(=O)(=O)c2c(F)c(F)c(F)c(F)c2F)c2ccc(C(=O)O)c(O)c2)cc(C2CC2)c1 ; CC(C)(C)c1cc(CN(C(=O)CN(Cc2ccc(Cl)cc2)S(=O)(=O)c2c(F)c(F)c(F)c(F)c2F)c2ccc(C(=O)O)cc2)cc(C2CC2)c1 ; O=C(O)c1ccc(N(Cc2cccc(C3CCCC3)c2)C(=O)CN(Cc2c(F)c(F)c(F)c(F)c2F)S(=O)(=O)c2c(F)c(F)c(F)c(F)c2F)cc1]

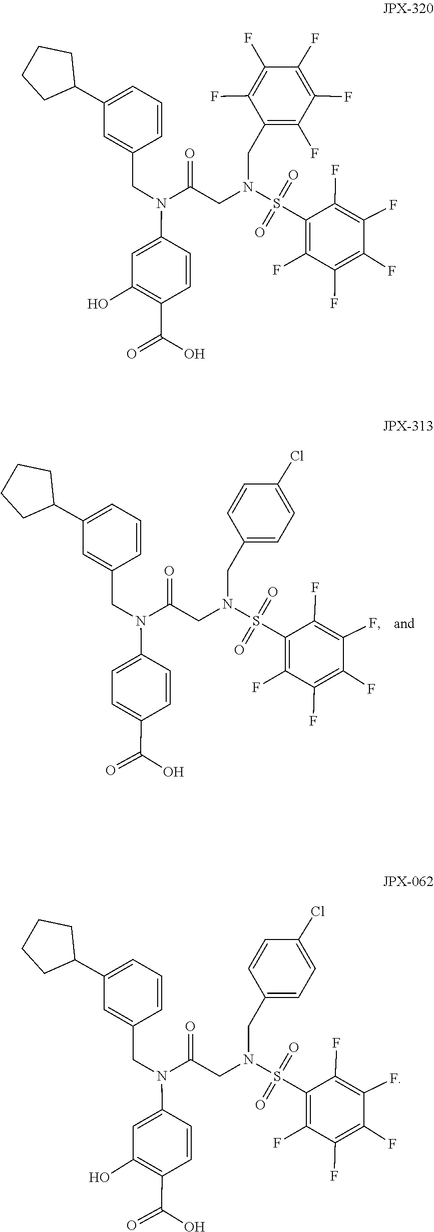 [SMILES: O=C(O)c1ccc(N(Cc2cccc(C3CCCC3)c2)C(=O)CN(Cc2c(F)c(F)c(F)c(F)c2F)S(=O)(=O)c2c(F)c(F)c(F)c(F)c2F)cc1O ; O=C(O)c1ccc(N(Cc2cccc(C3CCCC3)c2)C(=O)CN(Cc2ccc(Cl)cc2)S(=O)(=O)c2c(F)c(F)c(F)c(F)c2F)cc1 ; O=C(O)c1ccc(N(Cc2cccc(C3CCCC3)c2)C(=O)CN(Cc2ccc(Cl)cc2)S(=O)(=O)c2c(F)c(F)c(F)c(F)c2F)cc1O]

Methods of making Compounds of the Application

### General Methods

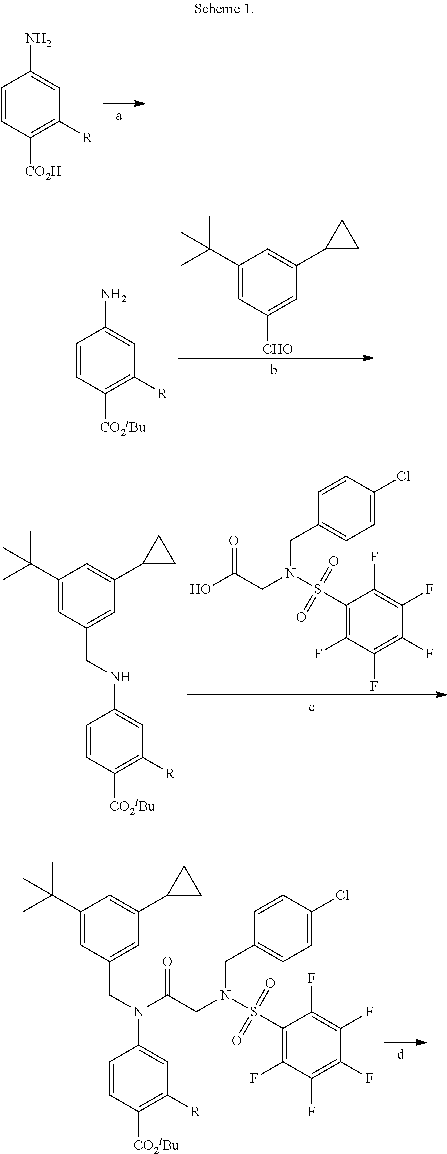

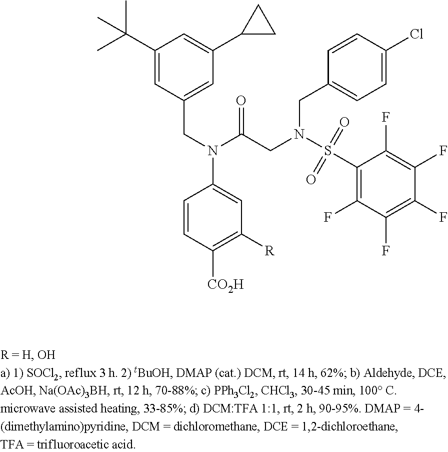

General Procedure a: ^{t}Butyl Esterification

4-aminosalicylic acid (1.0 eq.) was placed in a round bottom flask, followed by the dropwise addition of SOCl₂ (5.0 eq.) at rt. The reaction mixture was then refluxed for 3 h. Excess SOCl₂ was then removed under reduced pressure, and trace amounts by azeotrope with CHCl₃. 4-(dimethylamino)pyridine (0.1 eq.) and ^{t}BuOH (15 equiv) in DCM (1M) were added and the resulting mixture was stirred at rt for 14 h. The reaction was quenched by the addition of 1M NaOH and then extracted with EtOAc (4×). Combined organic fractions were washed with sat. NaHCO₃ (2×), sat. NaCl (1×) and dried over MgSO₄. The crude product was purified using Biotage Isolera automated column chromatographer and eluting with a gradient of Hexanes/EtOAc affording the primary aniline.

### General Procedure b: Reductive Amination Using Sodium Triacetoxy Borohodyride

To a solution of primary aniline (1.0 eq) and AcOH (1.1 eq) dissolved in anhydrous DCE (0.1 M) was added the corresponding aldehyde (1.0 eq). The solution was then stirred at rt for 10 mins after which Na(OAc)₃BH (1.5 eq) was added and the reaction allowed to stir at rt. Upon complete consumption of the primary aniline as indicated by TLC, the reaction was diluted with DCM and poured over a sat. solution of NaHCO₃. The layers were partitioned and aqueous layer was extracted with DCM (3×). Combined organic fractions were washed with brine, dried over MgSO₄ and concentrated in vacuo. The crude sample was absorbed directly onto silica for column chromatography purification using a gradient of hexanes and EtOAc affording the secondary aniline.

General Procedure c: Ph₃PCl₂ Peptide Coupling

To a stirred solution of the carboxylic acid (1.2 equiv) in CHCl₃ (0.08 M) was added Ph₃PCl₂ (2.5 equiv). The reaction mixture was stirred for 15 min or until complete dissolution at rt, followed by the dropwise addition of secondary aniline (1.0 equiv). The reaction mixture was then irradiated in a microwave at 100° C. for up to 45 min. The reaction mixture was cooled to 0° C. and quenched by the addition of sat. NaHCO₃. The two layers were partitioned and the aqueous layer was extracted with DCM (3×). Combined organic fractions were washed with sat. NaCl (1×), dried over MgSO₄. The crude sample was adsorbed directly onto silica and purified via column chromatography using an appropriate gradient of hexanes and EtOAc.

General Procedure d: t-Butyl Ester Deprotection

A solution of t-butyl ester (1 eq) was dissolved in 1:1 mixture of TFA and DCM (0.1 M) solution. The resultant solution was allowed to stir for 2 h and then co-evaporated with MeOH (3×) and CHCl₃ (3×).

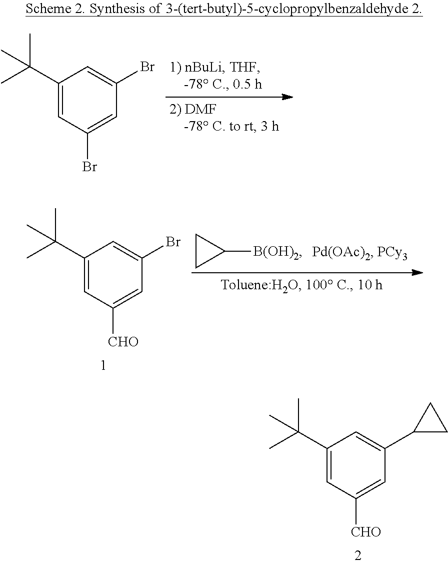

### 3-bromo-5-(tert-butyl)benzaldehyde (1)

A solution of 1,3-dibromo-5-(tert-butyl)benzene (2.57 mmol) in anhydrous THF (0.3M) was cooled to −78° C. followed by the dropwise addition of nBuLi (2.5M in hexane, 2.83 mmol) and stirred for 0.5 h at this temperature under N₂. DMF (3.85 mmol) was then slowly added and the reaction mixture was allowed to gradually warm from −78° C. to rt over 3 h. The reaction was quenched by the adding a saturated solution of NH₄Cl (20 mL). The two layers were partitioned and the aqueous layer was extracted with Et₂O (3×). Combined organic fractions were washed with brine, dried over MgSO₄ and concentrated in vacuo. Crude product 1 was isolated as a yellow oil (544 mg, 88%) and used directly in the following step.

¹H NMR (400 MHz, Chloroform-d) δ 9.95 (s, 1H), 7.83 (s, 1H), 7.82 (s, 1H) 7.77 (t, J=1.8 Hz, 1H), 1.36 (s, 9H).

### 3-(tert-butyl)-5-cyclopropylbenzaldehyde (2)

An oven dried round bottom flask equipped with a stirbar was charged with 1 (3.11 mmol), cyclopropylboronic acid (4.35 mmol), tricyclohexylphosphine (0.311 mmol) and K₃PO₄ (12.4 mmol) was purged with N₂. Toluene (0.2M) and H₂O (4M) were then added, followed by Pd(OAc)₂ (0.156 mmol) and the reaction mixture was placed in an oil bath at 100° C. and allowed to stir for 10 h. The reaction was the cooled back down to rt and filtered through celite and washed with EtOAc. The filtrate was diluted with EtOAc and H₂O and transferred to a separatory funnel. The two layers were partitioned and the aqueous layer was extracted with EtOAc (3×). Combined organic fractions were washed with brine and dried over MgSO₄. Crude material was directly adsorbed onto silica and purified using the Biotage Isolera automated column chromatography with a Hexanes/EtOAc gradient. 3-(tert-butyl)-5-cyclopropylbenzaldehyde was obtained as clear oil (452 mg, 72%).

¹H NMR (400 MHz, Chloroform-d) δ 9.97 (s, 1H), 7.69 (t, J=1.7 Hz, 1H), 7.44 (t, J=1.9 Hz, 1H), 7.34 (t, J=1.5 Hz, 1H), 2.00-1.93 (m, 1H), 1.35 (s, 7H), 1.06-0.96 (m, 2H), 0.79-0.70 (m, 2H). ¹³C NMR (101 MHz, CDCl₃) δ 192.96, 152.07, 144.85, 136.49, 129.91, 124.20, 123.50, 34.78, 31.25, 15.44, 9.49.

### tert-butyl 4-((3-(tert-butyl)-5-cyclopropylbenzyl)amino)-2-hydroxy-benzoate (3)

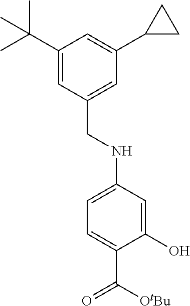 [SMILES: CC(C)(C)OC(=O)c1ccc(NCc2cc(C3CC3)cc(C(C)(C)C)c2)cc1O]

Compound 3 was prepared according to general procedure b, and was isolated as an amorphous white solid (78%). ¹HNMR (400 MHz, Chloroform-d) δ 11.39 (s, 1H), 7.65 (d, J=8.5 Hz, 1H), 7.22 (s, 1H), 7.15 (s, 1H), 6.91 (s, 1H), 6.19-6.15 (m, 2H), 4.50 (broad s, 1H), 4.33 (s, 2H), 2.00-1.93 (m, 1H), 1.66 (s, 9H), 1.39 (s, 9H), 1.05-0.98 (m, 2H), 0.79-0.75 (m, 2H). ¹³C NMR (101 MHz, CDCl₃) δ 170.11, 163.96, 153.96, 151.75, 144.23, 137.90, 131.48, 122.40, 122.07, 121.90, 105.32, 103.49, 98.07, 81.42, 48.13, 34.73, 31.47, 28.45, 15.68, 9.37.

### tert-butyl 4-(N-(3-(tert-butyl)-5-cyclopropylbenzyl)-2-ON-(4-chlorobenzyl)-2,3,4,5,6-pentafluorophenyl)sulfonamido) acetamido)-2-hydroxybenzoate (4)

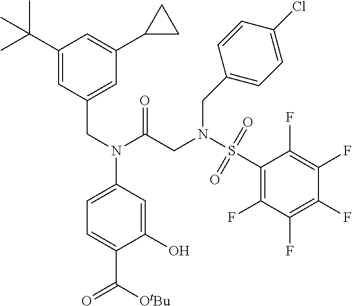 [SMILES: CC(C)(C)OC(=O)c1ccc(N(Cc2cc(C3CC3)cc(C(C)(C)C)c2)C(=O)CN(Cc2ccc(Cl)cc2)S(=O)(=O)c2c(F)c(F)c(F)c(F)c2F)cc1O]

Compound 4 was prepared according to general procedure c, and was isolated as an amorphous beige solid (66%). ¹HNMR (400 MHz, Chloroform-d) δ 11.07 (s, 1H), 7.65 (d, J=8.4 Hz, 1H), 7.28 (d, J=8.4 Hz, 2H), 7.18 (d, J=8.4 Hz, 2H), 7.03 (t, J=1.8 Hz, 1H), 6.81 (d, J=1.7 Hz, 1H), 6.59 (s, 1H), 6.37 (s, 1H), 6.21 (d, J=8.3 Hz, 1H), 4.68 (s, 2H), 4.62 (s, 2H), 3.81 (s, 2H), 1.89-1.83 (m, 1H), 1.60 (s, 9H), 1.24 (s, 9H), 0.99-0.94 (m, 2H), 0.65-0.61 (m, 2H). ¹³C NMR (101 MHz, CDCl₃) δ 168.80, 165.54, 162.57, 151.35, 145.64, 144.02, 135.36, 134.47, 132.82, 131.66, 130.10, 129.10, 123.16, 122.75, 122.52, 118.43, 116.91, 114.10, 83.73, 53.15, 50.53, 47.74, 34.51, 31.23, 28.13, 15.49, 9.24.

### tert-butyl 4-(N-(3-(tert-butyl)-5-cyclopropylbenzyl)-2-ON-(4-chlorobenzyl)-2,3,4,5,6-pentafluorophenyl)sulfonamido) acetamido)-2-hydroxybenzoate (compound I)

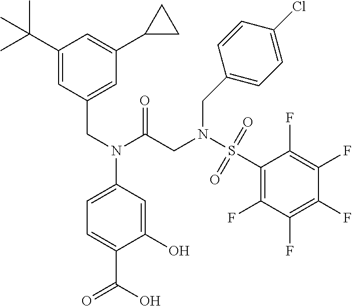 [SMILES: CC(C)(C)c1cc(CN(C(=O)CN(Cc2ccc(Cl)cc2)S(=O)(=O)c2c(F)c(F)c(F)c(F)c2F)c2ccc(C(=O)O)c(O)c2)cc(C2CC2)c1]

Compound I was prepared according to General Procedure d, and was isolated as an amorphous white powder (89%). ¹H NMR (400 MHz, Chloroform-d) δ 10.48 (s, 1H), 7.79 (d, J=8.4 Hz, 1H), 7.30-7.24 (d, J=8.2 Hz, 2H), 7.18 (d, J=8.2 Hz, 2H), 7.05 (s, 1H), 6.80 (s, 1H), 6.61 (s, 1H), 6.41 (s, 1H), 6.28 (s, 1H), 4.71 (s, 2H), 4.63 (s, 2H), 3.84 (s, 2H), 1.87 (ddd, J=13.6, 8.5, 5.1 Hz, 1H), 1.24 (s, 8H), 1.00-0.92 (m, 2H), 0.69-0.56 (m, 2H). HRMS (ESI+) Calculated for (C₃₆H₃₂Cl F₅N₂O₆S+H) 751.1668, found 751.1673.

### tert-butyl 4-((3-(tert-butyl)-5-cyclopropylbenzyl)amino)-2-hydroxybenzoate (6)

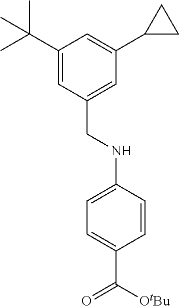 [SMILES: CC(C)(C)OC(=O)c1ccc(NCc2cc(C3CC3)cc(C(C)(C)C)c2)cc1]

Compound 6 was prepared according to General Procedure b, and was isolated as an amorphous beige solid (75%). ¹H NMR (400 MHz, Chloroform-d) δ 7.84 (d, J=8.6 Hz, 2H), 7.16 (d, J=1.6 Hz, 1H), 7.09 (d, J=1.8 Hz, 1H), 6.86 (d, J=1.7 Hz, 1H), 6.60 (d, J=8.8 Hz, 2H), 4.37 (s, 1H), 4.31 (s, 2H), 1.90 (tt, J=8.5, 5.1 Hz, 1H), 1.59 (s, 9H), 1.32 (s, 9H), 1.01-0.90 (m, 2H), 0.74-0.65 (m, 2H). ¹³C NMR (101 MHz, CDCl₃) δ 166.20, 151.73, 151.60, 144.18, 138.07, 131.34, 122.33, 122.00, 121.79, 120.54, 111.50, 79.85, 48.29, 34.69, 31.40, 28.36, 15.60, 9.27.

### tert-butyl 4-(N-(3-(tert-butyl)-5-cyclopropylbenzyl)-2-ON-(4-chlorobenzyl)-2,3,4,5,6-pentafluorophenyl)sulfonamido) acetamido)benzoate (7)

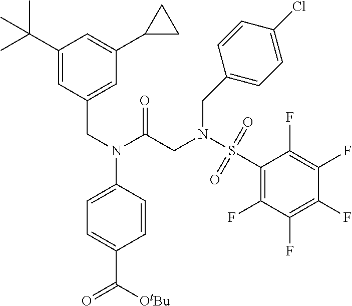 [SMILES: CC(C)(C)OC(=O)c1ccc(N(Cc2cc(C3CC3)cc(C(C)(C)C)c2)C(=O)CN(Cc2ccc(Cl)cc2)S(=O)(=O)c2c(F)c(F)c(F)c(F)c2F)cc1]

Compound 7 was prepared according to General Procedure c, and was isolated as an amorphous beige solid (31%). ¹H NMR (400 MHz, Chloroform-d) δ 7.88 (d, J=8.1 Hz, 2H), 7.28 (d, J=8.2 Hz, 2H), 7.18 (d, J=8.4 Hz, 2H), 7.04 (t, J=1.8 Hz, 1H), 6.80-6.70 (m, 3H), 6.59 (s, 1H), 4.71 (s, 2H), 4.65 (s, 2H), 3.73 (s, 2H), 1.91-1.85 (m, 1H), 1.60 (s, 9H), 1.24 (s, 9H), 0.99-0.95 (m, 2H), 0.65-0.61 (m, 2H).

### 4-(N-(3-(tert-butyl)-5-cyclopropylbenzyl)-2-ON-(4-chlorobenzyl)-2,3,4,5,6-pentafluorophenyl)sulfonamido)acetamido)benzoic acid (Compound II)

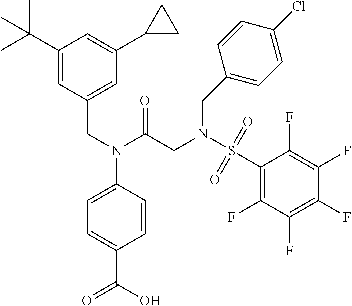 [SMILES: CC(C)(C)c1cc(CN(C(=O)CN(Cc2ccc(Cl)cc2)S(=O)(=O)c2c(F)c(F)c(F)c(F)c2F)c2ccc(C(=O)O)cc2)cc(C2CC2)c1]

Compound II was prepared according to General Procedure d, and was isolated as white amorphous powder (82%). ¹H NMR (400 MHz, Chloroform-d) δ 8.00 (d, J=8.0 Hz, 2H), 7.27 (d, J=8.3 Hz, 2H), 7.18 (d, J=8.3 Hz, 2H), 7.04 (d, J=1.8 Hz, 1H), 6.80 (d, J=8.1 Hz, 2H), 6.76 (s, 1H), 6.59 (d, J=1.7 Hz, 1H), 4.72 (s, 2H), 4.64 (s, 2H), 3.74 (s, 2H), 1.86 (tt, J=8.5, 5.1 Hz, 1H), 1.22 (s, 9H), 1.01-0.92 (m, 2H), 0.62 (dt, J=6.6, 4.7 Hz, 2H).

### 4-(N-(3-cyclopentylbenzyl)-2-((2,3,4,5,6-pentafluoro-N-((perfluorophenyl)methyl)phenyl)sulfonamido)acetamido)benzoic acid (JPX-303)

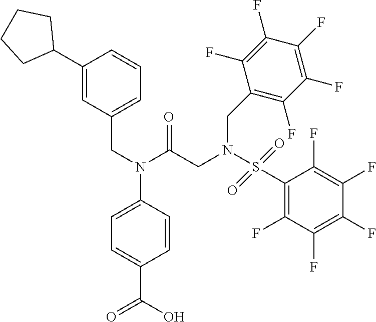 [SMILES: O=C(O)c1ccc(N(Cc2cccc(C3CCCC3)c2)C(=O)CN(Cc2c(F)c(F)c(F)c(F)c2F)S(=O)(=O)c2c(F)c(F)c(F)c(F)c2F)cc1]

Compound JPX-303 was prepared according to general procedure d, and was isolated as an amorphous white powder (88%). ¹H NMR (400 MHz, Chloroform-d) δ 8.08 (d, J=8.4 Hz, 2H), 7.21-7.14 (m, 2H), 7.05 (d, J=8.1 Hz, 2H), 6.88 (s, 1H), 6.85 (d, J=7.6 Hz, 1H), 4.81 (s, 2H), 4.79 (s, 2H), 3.88 (s, 2H), 2.91 (ddd, J=17.4, 9.7, 7.5 Hz, 1H) 2.03-1.96 (m, 2H), 1.78-1.71 (m, 2H), 1.69-1.64 (m, 2H), 1.52-143 (m, 2H). ¹³C NMR (101 MHz, CDCl₃) δ 170.10, 165.31, 147.07, 146.35, 145.68, 144.98, 144.69, 144.53, 144.19, 143.24, 142.31, 140.86, 138.53, 138.24, 137.07, 136.80, 135.28, 132.10, 129.76, 128.56, 128.35, 127.51, 126.74, 125.91, 116.02, 108.77, 53.42, 49.06, 45.69, 39.29, 34.47, 25.40.

### 4-(N-(3-cyclopentylbenzyl)-2-(2,3,4,5,6-pentalluoro-N-((perfluorophenyl)methyl)phenyl)sulfonamido)acetamido)-2-hydroxybenzoic acid (JPX-320)

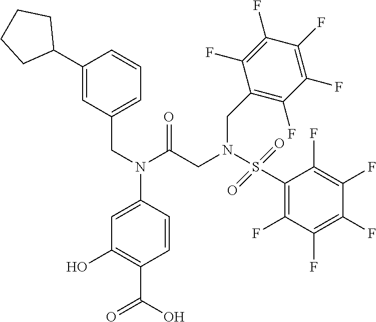 [SMILES: O=C(O)c1ccc(N(Cc2cccc(C3CCCC3)c2)C(=O)CN(Cc2c(F)c(F)c(F)c(F)c2F)S(=O)(=O)c2c(F)c(F)c(F)c(F)c2F)cc1O]

Compound JPX-320 was prepared according to general procedure d, and was isolated as an amorphous white powder (89%). ¹H NMR (400 MHz, Chloroform-d) δ 10.54 (s, 1H), 7.86 (d, J=8.4 Hz, 1H), 7.20-7.14 (m, 2H), 6.92-6.87 (m, 2H), 6.62 (s, 1H), 6.51 (d, J=8.4 Hz, 1H), 4.81 (s, 2H), 4.76 (s, 2H), 3.98 (s, 2H), 2.92 (q, J=7.2, 2.4 Hz, 1H), 2.02-1.99 (m, 2H), 1.77-1.74 (m, 2H), 1.69-1.65 (m, 2H), 1.52-1.46 (m, 2H).

### 4-(2-((N-(4-chlorobenzyl)-2,3,4,5,6-pentafluorophenyl)sulfonamido)-N-(3-cyclopentylbenzyl)acetamido)benzoic acid (JPX-313)

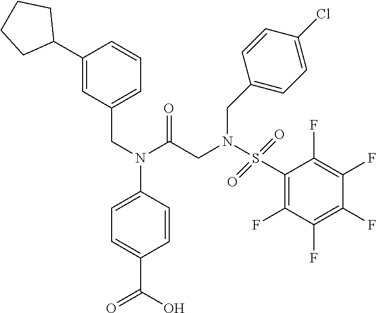 [SMILES: O=C(O)c1ccc(N(Cc2cccc(C3CCCC3)c2)C(=O)CN(Cc2ccc(Cl)cc2)S(=O)(=O)c2c(F)c(F)c(F)c(F)c2F)cc1]

Compound JPX-313 was prepared according to general procedure d, and was isolated as an amorphous white powder (92%). ¹H NMR (400 MHz, Chloroform-d) δ 7.98 (d, J=8.0 Hz, 2H), 7.26 (d, J=2.8 Hz, 1H), 7.20-7.17 (m, 4H), 6.87-6.85 (m, 4H), 4.72 (s, 2H), 4.64 (s, 2H), 3.74 (s, 2H), 2.94-2.89 (m, 1H), 2.02-1.99 (m, 2H), 1.77-1.74 (m, 2H), 1.68-1.65 (m, 2H), 1.50-1.47 (m, 2H).

### 4-(2-((N-(4-chlorobenzyl)-2,3,4,5,6-pentafluorophenyl)sulfonamido)-N-(3-cyclopentylbenzyl)acetamido)-2-hydroxybenzoic acid (JPX-062)

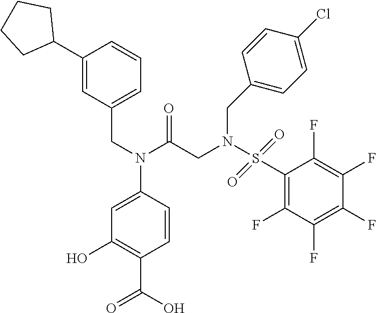 [SMILES: O=C(O)c1ccc(N(Cc2cccc(C3CCCC3)c2)C(=O)CN(Cc2ccc(Cl)cc2)S(=O)(=O)c2c(F)c(F)c(F)c(F)c2F)cc1O]

Compound JPX-062 was prepared according to general procedure d, and was isolated as an amorphous white powder (82%). ¹H NMR (400 MHz, Chloroform-d) δ 10.49 (s, 1H), 7.77 (d, 8.4 Hz, 1H), 7.29-7.27 (m, 2H), 7.21-7.15 (m, 4H), 6.90-6.87 (m, 2H), 6.45 (s, 1H), 6.32 (s, 1H), 4.71 (s, 2H), 4.63 (s, 2H), 2.95-2.90 (m, 1H), 2.03-1.99 (m, 2H), 1.78-1.75 (m, 2H), 1.69-1.65 (m, 2H), 1.51-1.48 (m, 2H).

The compounds of this application have unexpected metabolic stability to comparable compounds from literature.

### In Vitro Cell Viability Studies

Anti-cancer efficacy of exemplary compounds of this application was assessed in vitro in different cancer cell lines. Cell viability was examined following treatment at various concentration of inhibitor (0.097656-50 μM) using a cell Titer-Blue cell viability assay. 1×10⁴ cells/well were plated in 96-well assay plates in culture medium. All cells were grown in DMEM, IMDM and RPMI-1640 supplemented with 10% FBS. After 24 hrs, test compounds and vehicle controls were added to appropriate wells so the final volume was 100 μl in each well. The cells were cultured for the desired test exposure period (72 hrs) at 37° C. and 5% CO₂. The assay plates were removed from 37° C. incubator and 20 μl/well of CellTiter-Blue® Reagent was added. The plates were incubated using standard cell culture conditions for 1-4 hours and the plates were shaken for 10 seconds and fluorescence recorded at 560/590 nm.

Exemplary compounds of the application showed IC₅₀ values in the range of 0.4-5 μM against cancer cells, such as MV4-11, MOLM-13, and K562. The IC50 values for healthy cells such as MRC9 was typically greater than 20 μM.

Compound I and II were tested for their efficacy against selected chronic myelogenous leukemia, acute myeloid leukemia and healthy human lung cell lines using the protocol stated above.

Table 1 presents the IC₅₀ value of compound I against various cells lines.

TABLE 1
IC₅₀ values of compounds as described herein
against various cancer a healthy cell lines
IC50 (μM)

| | Type of cancer | | | | | |
|---|---|---|---|---|---|---|
| | | AML | | | | |
| | CML | MV-4- | MOLM- | NIH | Healthy cell line | |
| Cell line | K562 | 11 | 13 | 3T3 | HaCat | MRC-9 |
| Compound I | 4.1 | 0.56 | 1.7-2.5 | | 20 | 30 |
| Compound II | | 0.48 | | | | |
| JPX-303 | 2.6 | 0.21 | | 5.19 | | |
| JPX-320 | | 1.1 | 0.8 | | | |
| JPX-313 | | 0.93 | 1.1 | | | |
| JPX-062 | | 7.9 | 6.4 | | | |

The compounds of this application have unexpected improvements in anti-cancer efficacy over the comparable compounds from literature. As an example of the exemplary activity of Formula I class of compounds in acute myeloid leukemia cells (MV-4-11 cells), compound I and compound II have IC₅₀'s of 0.56 and 0.48 μM, respectively, compared to analogous compound, AC-3-19 (described in WO2015179956) which showed significantly lower efficacy with an IC₅₀ ˜3-5 μM.

### Pharmacokinetics—ADME Studies

### Intrinsic Clearance of Compounds I and II in Mouse Hepatocyte

In vitro T_{1/2} (min) for compound I was determined to be 100 mins.

A stock of 100 μM test compound was prepared by diluting the 10 mM test compound in DMSO with a solution of 50% acetonitrile and 50% water. In a 96-well non-coated plate, 198 μL of hepatocytes was pipetted, and the plate was placed in the incubator on an orbital shaker to allow the hepatocytes to warm for 10 minutes. To this solution was added 2 μL of the 100 μM test compound to start the reaction, and the plate was placed on an orbital shaker. At time points of 0, 15, 30, 60, 90 and 120 minutes, the aliquots were mixed with a solution of acetonitrile and internal standard (100 nM alprazolam, 200 nM labetalol, and 2 μM ketoprofen) to terminate the reaction. The reaction solution was then vortexed for 10 minutes and centrifuged at 4,000 rpm for 30 minutes at 4° C. 400 μL of the supernatant was transferred to one new 96-well plate, centrifuged at 4,000 rpm for 30 minutes at 4° C., and 100 μL of the supernatant was transferred to a new 96-well plate ensuring the pellet was not disturbed. 100 μL of ultrapure water was added to all samples for analysis by LC-MS/MS.

The in vitro half-life (T_{1/2}) was determined by the linear regression of the natural logarithm of the remaining percentage of the parent drug vs. incubation time curve. The slope value (k) of the curve was then substituted into the following equation to determine the in vitro half-life

$$in vitro {T}_{1 / 2} = - ( \frac{0.698}{k} )$$

The in vitro intrinsic clearance (in vitro CLᵢₙₜ, in μL/min/10⁶ cells) was determined by the following equation.

$$in vitro {CL}_{int} = ( \frac{0.698}{{T}_{\frac{1}{2}}} ) * ( \frac{volume of incubation}{number of hepatocytes} )$$

Where volume of incubation=0.2 mL and number of hepatocytes per well=0.1×10⁶ cells.

Bioanalytical method: Column—Phenomenex Synergi 4μ Hydro-PR 80A (2.0×30 mm).

Mobile phase—0.1% formic acid in water (solvent A) and 0.1% formic acid in acetonitrile (solvent B). Column temperature—room temperature. Injection volume—10 μL. MS analysis—API 4000 instrument from AB Inc (Canada) with an ESI interface.

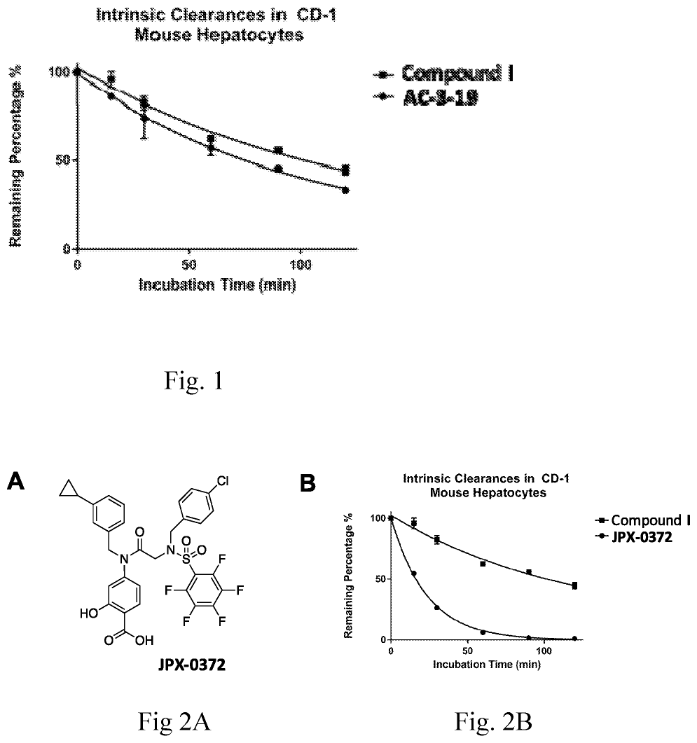

As can be appreciated from FIG. 1, compound I has T_{1/2} of 101 min, while pentafluorobenzenesulfonamide containing AC-3-19 has a T_{1/2} of 83 min. Compound I exhibits slower clearance rates than the comparable compound from literature. As an example of the exemplary stability of Formula I class of compounds, analogous compound, JPX-0372 (structure shown in FIG. 2A), where the tert-butyl group is omitted, showed significantly faster clearance with a T_{1/2} of 16.4 mins (FIG. 2B) (Pharmaron, China).

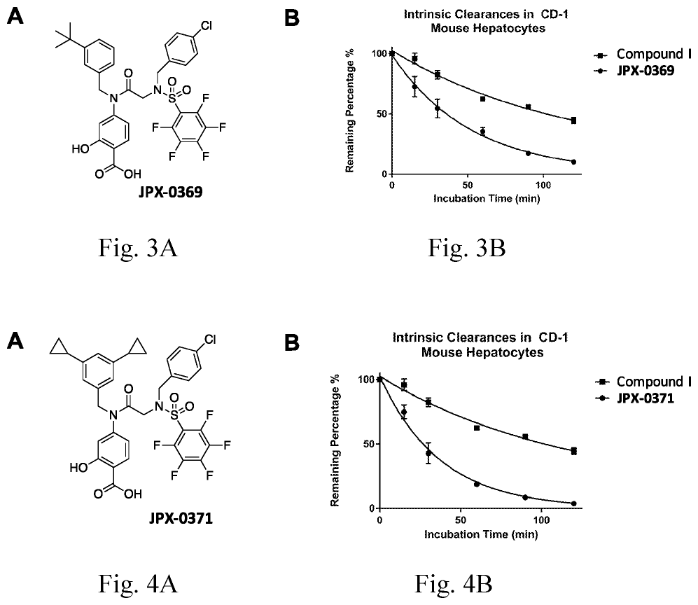

As a further example of metabolic stability afforded by compounds of Formula I, compound JPX-0369 (FIG. 3A), where the c-Pr group of compound I was removed, showed a significantly faster clearance rate of T_{1/2}=36.5 mins (FIG. 3B).

As a further example of metabolic stability afforded by compounds of Formula I, compound JPX-0371 (FIG. 4A), possessing a symmetrically disubstituted 3,5-di-c-Pr group instead of the asymmetrically di-substituted compound I, again showed a much inferior clearance rate of T_{1/2}=25.1 mins (FIG. 4B).

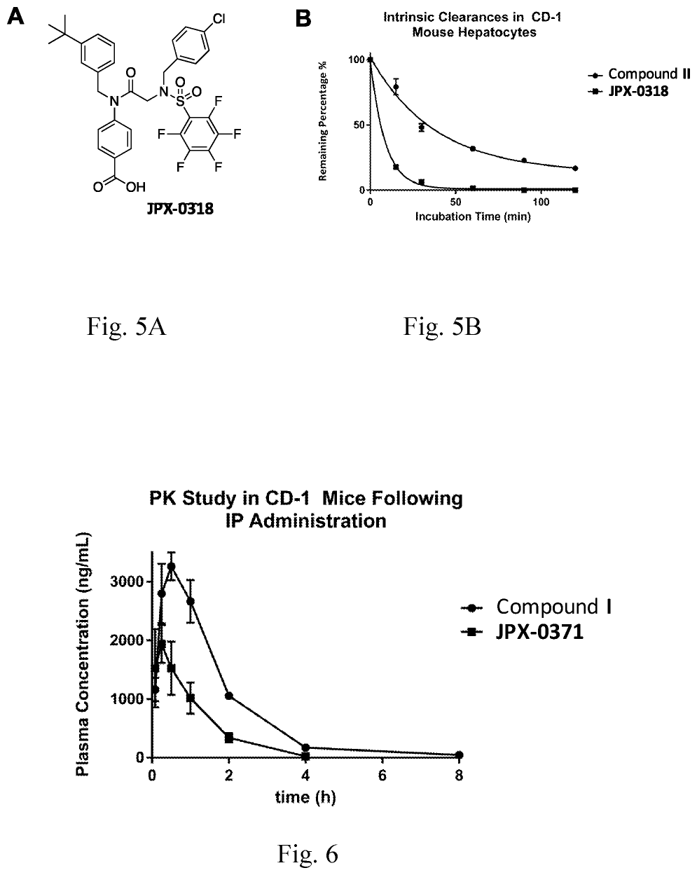

As a further example of metabolic stability afforded by compounds of Formula I, compound JPX-0318 (FIG. 5A), possessing a benzoic acid and mono-substituted 3-tert-butyl group instead of the asymmetrically di-substituted compound II possessing a benzoic acid, again showed a much inferior clearance rate of T_{1/2}=10.3 mins as compared to T_{1/2}=46 min for compound II (FIG. 5B).

As can thus be appreciated, compounds of formula I as described herein possess superior clearance rates in mouse hepatocytes to previous examples of pentafluorobenzenesulfonamide-containing compounds.

### Bioavailability Studies in CD-1 Mice (IP Injection).

The study groups for PK for compound I, II and comparative experiments with AC-3-19 and JPX-0371 experiments are shown in Table 2.

TABLE 2

| | | Dose Level | Conc. | Dose Volume | Administration | No. of |
|---|---|---|---|---|---|---|
| Group | Treatment | (mg/kg) | (mg/mL) | (mL/kg) | Route | Animals |
| 1 | Compound I | 20 | 4 | 5 | IP | 3M |
| 2 | Compound II | 20 | 4 | 5 | IP | 3M |
| 3 | AC-3-19 | 20 | 4 | 5 | IP | 3M |
| 4 | JPX-0371 | 20 | 4 | 5 | IP | 3M |
| 5 | JPX-303 | 20 | 4 | 5 | IP | 3M |
| 6 | JPX-320 | 20 | 4 | 5 | IP | 3M |
| 7 | JPX-313 | 20 | 4 | 5 | IP | 3M |
| 8 | JPX-062 | 20 | 4 | 5 | IP | 3M |

All animals had free access to food and water. Dose formulation processing during dosing: The formulations will be kept stirred at room temperature for at least 15 min before dosing and during the dosing. Pharmacokinetics (PK) Schedule shown below in Table 3.

TABLE 3

| Group | PK time points |
|---|---|
| IP | Plasma: 5, 15, 30 min, 1, 2, 4, 8 and 24 hours post dose |

Approximately 0.03 mL blood was collected at each time point. Blood of each sample was transferred into plastic micro centrifuge tubes containing heparin-Na as anticoagulant. Collection tubes with blood samples and anticoagulant were inverted several times for proper mixing of the tube contents and then placed on wet ice prior to centrifugation for plasma. The blood samples were centrifuged at 4000 g for 5 min at 4° C. to obtain plasma. The samples were stored in a freezer at −75±15° C. prior to analysis. The study used CD-1 mice (male), n=3, at age approx. 6-8 weeks (20-30 g).

### Dose Formulation

TABLE 4

| | |
|---|---|
| Formulation Frequency: | Freshly prepared on the day of dosing |
| Vehicle Composition: | IP: 10% DMA/65% PEG400/25% Saline |
| Storage Condition: | Dose formulation for dosing: Room |
| | temperature |

Concentrations of compounds in the plasma samples were analyzed using a LC-MS/MS method. WinNonlin (Phoenix™, version 6.1). Other similar software could have also been used for pharmacokinetic calculations. Pharmacokinetic parameters were calculated, whenever possible from the plasma concentration versus time data: IP parameters including T_{1/2}, Cₘₐₓ, Tₘₐₓ, AUCₗₐₛₜ, AUC_{inf}, MRT are calculated.

FIG. 6 shows the clearance rate of compounds I and comparative compound, JPX-0371. Compound I has a calculated T_{1/2} of 3.9 hours, while pentafluorobenzenesulfonamide containing JPX-0371 has a T_{1/2} of 0.66 hours. The compounds of this application have unexpectedly much slower clearance rates, higher bioavailability than the comparable analogous compounds as shown in Tables 5 and 6 where PK parameters are outline for compound I (Table 5) and JPX-0371 (Table 6).

TABLE 5
PK data for compound I

| | No pts | | | | | | AUC | | |
|---|---|---|---|---|---|---|---|---|---|
| | used | | tₘₐₓ | Cₘₐₓ | AUCₗₐₛₜ | AUC_{Inf} | Extr | MRT | AUC/D |
| Animal | for t_{1/2} | t_{1/2}(h) | (h) | (ng/mL) | (h*ng/mL) | (h*ng/mL) | (%) | (h) | (h*mg/mL) |
| Mouse 1 | 3.00 | 3.92 | 0.500 | 3010 | 5803 | 5823 | 0.346 | 1.87 | 290 |
| Mouse 2 | 3.00 | 3.91 | 0.500 | 3290 | 7217 | 7245 | 0.385 | 1.99 | 361 |
| Mouse 3 | 3.00 | 3.97 | 0.500 | 3480 | 6545 | 6566 | 0.325 | 1.81 | 327 |
| N | 3 | 3 | 3 | 3 | 3 | 3 | 3 | 3 | 3 |
| Mean | 3.00 | 3.93 | 0.500 | 3260 | 6522 | 6545 | 0.352 | 1.89 | 326 |
| SD | 0.000 | 0.03 | 0.000 | 236 | 708 | 711 | 0.030 | 0.09 | 35 |
| CV % | 0.000 | 0.785 | 0.000 | 7.25 | 10.8 | 10.9 | 8.65 | 4.90 | 10.8 |

TABLE 6
PK data for JPX-0371

| | No pts | | | | | | AUC | | |
|---|---|---|---|---|---|---|---|---|---|
| | used for | | tₘₐₓ | Cₘₐₓ | AUCₗₐₛₜ | AUC_{Inf} | Extr | MRT | AUC/D |
| Animal | t_{1/2} | t_{1/2}(h) | (h) | (ng/mL) | (h*ng/mL) | (h*ng/mL) | (%) | (h) | (h*mg/mL) |
| Mouse 1 | 3.00 | 0.574 | 0.250 | 1600 | 1895 | 1912 | 0.875 | 0.911 | 94.7 |
| Mouse 2 | 3.00 | 0.503 | 0.500 | 2050 | 2830 | 2845 | 0.528 | 0.920 | 142 |
| Mouse 3 | 3.00 | 0.906 | 0.083 | 2290 | 2709 | 2713 | 0.157 | 1.02 | 135 |
| N | 3 | 3 | 3 | 3 | 3 | 3 | 3 | 3 | 3 |
| Mean | 3.00 | 0.661 | 0.278 | 1980 | 2478 | 2490 | 0.520 | 0.95 | 124 |
| SD | 0.00 | 0.215 | 0.210 | 350 | 509 | 505 | 0.359 | 0.06 | 25 |
| CV % | 0.000 | 32.5 | 75.6 | 17.7 | 20.5 | 20.3 | 69.1 | 6.21 | 20.5 |

Similarly, the t_{1/2}(hr) obtained for compounds JPX-303, JPX-320, JPX-313, and JPX-062 are respectively of 0.35, 0.861, 0.31, and 0.94.

It will be appreciated that the amount of a compound of the invention required for use in treatment will vary not only with the particular compound selected but also with the route of administration, the nature of the condition for which treatment is required and the age and condition of the patient and will be ultimately at the discretion of the attendant physician. Generally, the amount administered will be empirically determined, typically in the range of about 10 μg to 100 mg/kg body weight of the recipient.

The desired dose may conveniently be presented in a single dose or as divided dose administered at appropriate intervals, for example as two, three, four or more doses per day.

Pharmaceutical compositions include, without limitation, those suitable for oral, (including buccal and sub-lingual), transdermal, or parenteral (including intramuscular, sub-cutaneous and intravenous) administration or in a form suitable for administration by inhalation.

The formulations may, where appropriate, be conveniently presented in discrete dosage units and may be prepared by any of the methods well known in the art of pharmacy. The methods for preparing a pharmaceutical composition can include the steps of bringing into association the compound as defined herein and pharmaceutically acceptable excipients and then, if necessary, shaping the product into the desired formulation, including applying a coating when desired.

Pharmaceutical compositions suitable for oral administration may conveniently be presented as discrete units such as capsules, cachets or tablets each containing a predetermined amount of the active ingredient; as a powder or granules; as a solution, a suspension or as an emulsion. Tablets and capsules for oral administration may contain conventional excipients such as binding agents, fillers, lubricants, disintegrants, or wetting agents. The tablets may be coated according to methods well known in the art. Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), or preservatives.

The compounds and combinations as defined herein may also be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulatory agents such as suspending, stabilizing and/or dispersing agents. Alternatively, the active ingredient may be in powder form, obtained by aseptic isolation of sterile solid or by lyophilisation from solution, for constitution with a suitable vehicle, e.g. sterile water or saline, before use.

Compositions suitable for topical administration in the mouth include lozenges comprising the active ingredient in a flavoured base, usually sucrose and acacia or tragacanth; pastilles comprising the active ingredient in an inert base such as gelatin and glycerin or sucrose and acacia; and mouthwashes comprising the active ingredient in a suitable liquid carrier.

For administration by inhalation, the compounds and combinations as defined herein may take the form of a dry powder composition, for example a powder mix of the compound and a suitable powder base such as lactose or starch. The powder composition may be presented in unit dosage form in, for example, capsules or cartridges or e.g. gelatin or blister packs from which the powder may be administered with the aid of an inhalator or insufflator.

These compounds being STAT3/STAT5 inhibitors much like those described in WO2013/177534, it is anticipated that the compounds described herein will have the same utility with a similar or higher activity for treating cancer such as for example pancreatic cancer, multiple myeloma, brain cancer, and breast cancer, while having a longer clearance rate, making these compounds better drug candidates.

While the disclosure has been described in connection with specific embodiments thereof, it is understood that it is capable of further modifications and that this application is intended to cover any variation, use, or adaptation of the disclosure following, in general, the principles of the disclosure and including such departures from the present disclosure that come within known, or customary practice within the art to which the disclosure pertains and as may be applied to the essential features hereinbefore set forth, and as follows in the scope of the appended claims.

1. A compound of formula I: 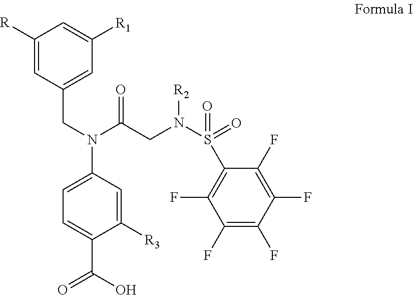
or a pharmaceutically acceptable salt, solvate or hydrate thereof, wherein 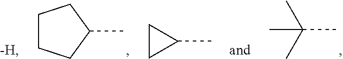
Wherein when one of R and R₁ is —H, the other of R and R₁ is a cyclopentyl moiety,
R₂ is a benzyl substituted with 1-5 halogens, and
R₃ is selected from the group consisting of —H or —OH.

2. The compound of claim 1, or a pharmaceutically acceptable salt, solvate or hydrate thereof, wherein R₂ is a 4-Chloro-benzyl group or a 4-bromo-benzyl group.

3. A pharmaceutical composition comprising a compound as defined in any claim 1 or 2 or a pharmaceutically acceptable salt, solvate or hydrate thereof, and an acceptable excipient.

4. A method of treating a cell proliferative disorder comprising administering a compound as defined in claim 1 or 2, or pharmaceutically acceptable salts and/or solvates thereof, to a subject in need thereof.

5. The method of claim 4, wherein the cell proliferative disorder is cancer.

6. The method of claim 5, wherein the cancer is a cancer associated with pSTAT3 and/or pSTAT5 overexpression.

7. The method of claim 6, wherein the cancer is a hematological cancer or a brain cancer.

8. The method of claim 7, wherein the cancer is acute myelomoid leukemia, chronic myelogenous leukemia or medulloblastoma.

9. A method for inhibiting STAT3 and/or STAT5 activity, comprising administering a therapeutically effective amount of a compound as defined in claim 1 or 2 or a pharmaceutically acceptable salt, solvate or hydrate thereof, to a patient.

10. A method for treating or preventing a cancer with cancer cells harbouring overexpression of pSTAT3 and/or pSTAT5, comprising administering a therapeutically effective amount of a compound as defined in claim 1 or 2, or a pharmaceutically acceptable salt, solvate or hydrate thereof to a patient.

11. The method of claim 10, where said cancer is from solid or hematological tumors.

12. The method of claim 11, wherein said cancer is selected from the group consisting of breast cancer, brain cancer, liver cancer, prostate cancer, pancreatic cancer, blood cancer, skin cancer, head cancer, neck cancer, glioblastoma, multiple myeloma, acute myelogenic leukemia (AML) and acute lymphoblastic leukemia.

13. Use of a compound as defined in claim 1 or 2, or pharmaceutically acceptable salts and/or solvates thereof, for treating a cell proliferative disorder in a subject in need thereof.

14. The use of claim 13, wherein the cell proliferative disorder is cancer.

15. The method of claim 5, wherein the cancer is a cancer associated with pSTAT3 and/or pSTAT5 overexpression.

16. The use of claim 14, wherein the cancer is a hematological cancer or a brain cancer.

17. The use of claim 16, wherein the cancer is acute myelomoid leukemia, chronic myelogenous leukemia or medulloblastoma.

18. Use of a compound as defined in claim 1 or 2 or a pharmaceutically acceptable salt, solvate or hydrate thereof, for inhibiting STAT3 and/or STAT5 activity.

19. Use of a compound as defined in claim 1 or 2 or a pharmaceutically acceptable salt, solvate or hydrate thereof for treating a cancer with cancer cells harbouring activated STAT3 or STAT5.

20. The use of claim 19, wherein said cancer is from solid or hematological tumors.

21. The use of claim 20, wherein said cancer is selected from the group consisting of breast cancer, brain cancer, liver cancer, prostate cancer, pancreatic cancer, blood cancer, skin cancer, head cancer, neck cancer, glioblastoma, multiple myeloma, acute myelogenic leukemia (AML) and acute lymphoblastic leukemia.

22. Use of a composition as defined in claim 3, or pharmaceutically acceptable salts and/or solvates thereof, for treating a cell proliferative disorder in a subject in need thereof.

23. The use of claim 22, wherein the cell proliferative disorder is cancer.

24. The use of claim 23, wherein the cancer is a hematological cancer or a brain cancer.

25. The use of claim 24, wherein the cancer is acute myelomoid leukemia, chronic myelogenous leukemia or medulloblastoma.

26. The pharmaceutical composition as defined in claim 3 for use in inhibiting STAT3 and/or STAT5 activity.

27. The pharmaceutical composition as defined in claim 3 for use in treating a cancer with cancer cells harbouring activated STAT3 and/or STAT5.

28. The pharmaceutical composition as defined in claim 27, wherein said cancer is from solid or hematological tumors.

29. The pharmaceutical composition as defined in claim 28, wherein said cancer is selected from the group consisting of breast cancer, brain cancer, liver cancer, prostate cancer, pancreatic cancer, blood cancer, skin cancer, head cancer, neck cancer, glioblastoma, multiple myeloma, acute myelogenic leukemia (AML) and acute lymphoblastic leukemia.
